SEQUENCE LISTING (8 sequences)

SEQ ID NO: 1 - protein, 7 aa, synthetic construct
SNTSESF

SEQ ID NO: 2 - protein, 16 aa, synthetic construct
SNTSESFGSGGGSGGC

SEQ ID NO: 3 - protein, 12 aa, synthetic construct
NYSKPTDRQYHF

SEQ ID NO: 4 - protein, 22 aa, synthetic construct
SNTSESFKFRVTQLAPKAQIKE

SEQ ID NO: 5 - protein, 8 aa, synthetic construct
GSGGGSGG

SEQ ID NO: 6 - protein, 9 aa, synthetic construct
GSGGGSGGC

SEQ ID NO: 7 - protein, 190 aa, synthetic construct
MSTVGTGKLTRAQRRAAARKNKRNTRVVQPVIVEPIASGQGKAIKAWTGYSVSKWTASCAAAEAKVTSAITISLPNELSSERNKQLKVGRVLLWLGLLPSVSGTVKSCVTETQTTAAASFQVALAVADNSKDVVAAMYPEAFKGITLEQLTADLTIYLYSSAALTEGDVIVHLEVEHVRPTFDDSFTPVY

SEQ ID NO: 8 - protein, 374 aa, synthetic construct
MEQNLFALSLDDTSSVRGSLLDTKFAQTRVLLSKAMAGGDVLLDEYLYDVVNGQDFRATVAFLRTHVITGKIKVTATTNISDNSGCCLMLAINSGVRGKYSTDVYTICSQDSMTWNPGCKKNFSFTFNPNPCGDSWSAEMISRSRVRMTVICVSGWTLSPTTDVIAKLDWSIVNEKCEPTIYHLADCQNWLPLNRWMGKLTFPQGVTSEVRRMPLSIGGGAGATQAFLANMPNSWISMWRYFRGELHFEVTKMSSPYIKATVTFLIAFGNLSDAFGFYESFPHRIVQFAEVEEKCTLVFSQQEFVTAWSTQVNPRTTLEADGCPYLYAIIHDSTTGTISGDFNLGVKLVGIKDFCGIGSNPGIDGSRLLGAIAQ

(19) United States
(12) Patent Application Publication
(10) Pub. No.: US 2025/0127842 A1
(43) Pub. Date: Apr. 24, 2025

(54) IMMUNE CHECKPOINT TARGETING THERAPEUTIC NANOPARTICLES

(71) Applicant: The Regents of the University of California, Oakland, CA (US)

(72) Inventors: Nicole F. Steinmetz, La Jolla, CA (US); Chao Wang, La Jolla, CA (US)

(21) Appl. No.: 18/686,840

(22) PCT Filed: Aug. 29, 2022
(86) PCT No.: PCT/US2022/041925

§ 371 Date: Feb. 26, 2024

Related U.S. Application Data
(60) Provisional application No. 63/238,725, filed on Aug. 30, 2021.

(51) Int. Cl. A61K 38/08; A61K 38/10; B82Y 5/00; C07K 7/06; C07K 7/08; C12N 1/20; C12N 5/04; C12N 7/00; C12N 15/62

(52) U.S. Cl. CPC A61K 38/08; A61K 38/10; B82Y 5/00; C07K 7/06; C07K 7/08; C12N 1/20; C12N 5/04; C12N 7/00; C12N 15/62

(57) ABSTRACT

Provided herein are nanoparticles comprising peptides that target checkpoint inhibitors such PD-1 as well as methods for their use. Peptides may be conjugated to nanoparticles such as cowpea mosaic virus or a cowpea chlorotic mottle virus. Nanoparticles provided herein display efficacy against tumors in a mouse model of disseminated and aggressive ovarian cancer.

### CROSS-REFERENCE TO RELATED APPLICATION

This application is a U.S. 371 National Stage Application of PCT International Application No, PCT/US2022/041925, filed Aug. 29, 2022, which claims priority under 35 U.S.C. § 119 (e) to U.S. Provisional Application No. 63/238,725, filed Aug. 30, 2021, the contents of which are incorporated herein by reference in its entirety.

### STATEMENT OF GOVERNMENT SUPPORT

This invention was made with government support under Grant Nos. CA218292; CA224605; and CA253615 awarded by the National Institutes of Health (NIH). The government has certain rights in the invention.

### SEQUENCE LISTING

The instant application contains a Sequence Listing which has been submitted electronically in XML file format and is hereby incorporated by reference in its entirety. Said XML copy, created on Oct. 11, 2022, is named 114198-9040.xml is 9 kilobytes in size.

### BACKGROUND

The immune system plays a critical role in tumor surveillance and cancer immunotherapy is a recognized pillar of cancer therapy. Aggressive tumors present with an immunosuppressed tumor microenvironment (TME) which hinders intrinsic anti-tumor 40 immunity [1]. Immunotherapies targeting the TME and the various checkpoints of the cancer immunity cycle to modulate the activity of the immune system to promote its anti-tumor functions hold promise in cancer therapy, in particular targeting immunologically cold tumors [2]. Immune checkpoint inhibitors targeting the PD-1/PD-L1 axis overcome inhibition of effector T cell function and show promise as mono- and combination therapy. While immune checkpoint blockade has produced remarkable clinical outcomes for patients [3], most patients do not respond optimally or develop resistance [4]. Strategic immuno-combination therapies are the formula for success; more than 800 registered oncology trials focus on combination therapies.

A particularly powerful approach is the combination of in situ vaccination strategies with immune checkpoint blockade. The in situ vaccination strategies makes use of immunostimulatory agents administered directly into an identified tumor; the immunostimulatory agent acts as adjuvant to recruit and activate innate immune cells and the patient's tumor provide the source of antigen. Immune cell mediated tumor cell death releases tumor-associated antigens (TAAs) for processing by the innate immune cells which become antigen-presenting cells (APCs) to then prime the adaptive arm and leading to systemic anti-tumor immunity and memory [5].

### SUMMARY OF THE DISCLOSURE

Provided herein is a nanoparticle comprising a cowpea mosaic virus (CPMV) or a cowpea chlorotic mottle virus (CCMV) and a peptide that binds to an immune checkpoint or its ligand such as PD-1 or PD-L1. Any peptide that will bind the immune checkpoint or its ligand, e.g. PD-1 or PD-L1, can be used and these are known in the art. A non-limiting example of such comprises, or consists essentially of, or yet further consists of SNTSESF (SEQ ID NO: 1) with an optional linker, the linker comprising, consisting essentially of, or yet further consisting of GSGGGSGG (SEQ ID NO: 5) with an optional carboxy-terminal cysteine residue. In a further aspect, the peptide comprises, or consists essentially of, or yet further consists of SNTSESF (SEQ ID NO: 1) or an equivalent thereof having at least 70% identical, or at least 80%, or at least 90% or at least 95% sequence identity while still binding PD-1. In one aspect, the peptide or equivalent further comprises an optional linker, the linker comprising, consisting essentially of, or yet further consisting of GSGGGSGG (SEQ ID NO: 5) or an equivalent thereof having at least 70% identical, or at least 80%, or at least 90% or at least 95% sequence identity to GSGGGSGG (SEQ ID NO: 5), with an optional carboxy-terminal cysteine residue. In one aspect, the CPMV has an exposed lysine side chain. In one aspect, the lysine side chain is conjugated to an N-hydroxysuccinimide (NHS) ester and the maleimide of a maleimide-polyethylene glycols is conjugated with the c-terminal cysteine of the peptide. In another aspect, the peptide does further comprise the linker and the carboxy-terminal cysteine residue.

The nanoparticles can further comprise a detectable or a purification maker.

In one aspect, the nanoparticle has an average diameter of from about 10 to about 50 nm.

The number of peptides joined to the CPMV or CCMV can be 1 or more, or 2 or more or 5 or more, or 10 or more, or 15 or more, or 20 or more, or 25 or more, or 30 or more, or 35 or more, or 50 or more, or 100 or more, or 150 or more, or 200 or more, and ranges in between.

One embodiment of the disclosure relates to a nanoparticle comprising, or alternatively consisting essentially of, or yet further consisting of a cowpea mosaic virus (CPMV) or CCMV virus and the amino acid of SEQ ID NO: 1 or 2, or an equivalent thereof of at least 70% identical, or at least 80%, or at least 90% or at least 95% or similar to SEQ ID NO: 1 or 2, wherein the equivalent and a peptide recognize and binds PD-1 or PD-L1. In one aspect, the CPMV or CCMV has an exposed lysine side chain. In one aspect, the lysine side chain is conjugated to an N-hydroxysuccinimide (NHS) ester and the maleimide of a maleimide-polyethylene glycols is conjugated with the c-terminal cysteine of the peptide. In another aspect the peptide optionally comprises, or alternatively consists essentially of, or yet further consists of a c-terminal cysteine. In one aspect, the peptide does further comprise the linker. In another aspect, the peptide does further comprise the linker and the carboxy-terminal cysteine residue. The nanoparticles can further comprise a detectable or a purification maker.

In one aspect, the nanoparticle has an average diameter of from about 10 to about 50 nm.

The number of peptides joined to the CPMV or CCMV can be 1 or more, or 2 or more or 5 or more, or 10 or more, or 15 or more, or 20 or more, or 25 or more, or 30 or more, or 35 or more, or 50 or more, or 100 or more, or 150 or more, or 200 or more, and ranges in between.

The nanoparticles are useful in the methods as disclosed herein.

Provided herein are isolated polynucleotides encoding the nanoparticle of this disclosure or an equivalent thereof. The polynucleotide can be DNA or RNA. In one aspect, as provided herein, is a vector, comprising, or alternatively consisting essentially of, or yet further consisting of the polynucleotide of this disclosure. In a further aspect, the polynucleotide is linked to regulatory elements (promoter or enhancer for example) for replication or expression of the polynucleotide. For the production of vectors, the vector genome is expressed from a DNA construct encoding it in a host cell. In one aspect, as provided herein, is a host cell, comprising, or alternatively consisting essentially of, or yet further consisting of the polynucleotide of this disclosure. The polynucleotides, vectors and host cells can further comprise a detectable label.

Also provided is a method of producing the polynucleotide and nanoparticle by growing the cells containing the polynucleotide under conditions that favor replication and expression as desired. These conditions are known in the art.

Further provided herein, are a plurality of nanoparticles, where the nanoparticles are the same or different from each other. For example, the plurality can comprise the same or different peptide, diameter or linkers, as desired.

In one aspect, provided herein is a composition comprising, or alternatively consisting essentially of, or yet further consisting of the nanoparticle, polynucleotide, vector and/or host cell. In another aspect, provided herein is a composition comprising, or alternatively consisting essentially of, or yet further consisting of a carrier and one or more of the nanoparticle, polynucleotide, vector and/or the host cell of this disclosure.

In a further aspect, provided herein is a method for inducing an immune response the method comprising, or alternatively consisting essentially of, or yet further consisting of administering to the subject one or more of the nanoparticle, polynucleotide, vector, the composition and/or the host cell of this disclosure. Methods for detecting and monitoring an immune response are known in the art.

Also provided herein is a method for inhibiting the growth of a cancer cell that optionally expresses an immune checkpoint, e.g., PD-1, comprising, or alternatively consisting essentially of, or yet further consisting of contacting the cell with the nanoparticle, polynucleotide, vector, the composition and/or the host cell of this disclosure. As is apparent to the skilled artisan, the peptide used for the method must correspond to the immune checkpoint expressed on the cell, e.g., an anti-PD-1 peptide for a cell expressing PD-1. In one aspect, an additional therapy is contacted with the cell. The contacting can be concurrently or simultaneous. The contacting can be in vitro or in vivo. Methods to determine when cell growth has been inhibited are known in the art. In vitro, the method can be used to screen for combination therapies or for personalized treatments for animals, mammals and human.

In one aspect, the cancer cell is any of the cancer cells described herein, including primary and metastatic cells. In vivo, the method comprises administering to the subject an effective amount of the composition as described herein and can be used to test for new drug combinations (in an animal model) or for therapy itself. The treatment can be combined with other therapies and therefore can be a first line, second line, third line, fourth line or fifth line therapy. Methods to determine effectiveness of the therapy include reduction in tumor size or burden, prolonged progression-free survival, prolonged overall survival and reduced toxicity. These methods can be combined with other clinical markers, e.g., a reduction in a tumor marker, e.g., CA125 or methods to determine the identity of the checkpoint expressed by the cancer or tumor.

One embodiment of the disclosure relates to a method for treating cancer, optionally a cancer expressing an immune checkpoint, e.g., PD-1, in a subject in need thereof, comprising, or alternatively consisting essentially of, or yet further consisting of administering to the subject one or more of the nanoparticle, polynucleotide, vector, the composition and/or the host cell of this disclosure. As is apparent to the skilled artisan, the peptide used for the method must correspond to the checkpoint expressed on the cell, e.g., an anti-PD-1 peptide for a cell expressing PD-1. In one aspect, an additional therapy is administered to the subject. The administration can be concurrently or simultaneous.

In one aspect, the cancer is any of the cancer cells described herein, including primary and metastatic cancer. When practiced in a non-human subject or a human subject, the method comprises administering an effective amount of the composition as described herein. In an animal model the method can be used to test for new drug combinations or for therapy itself. The treatment can be combined with other therapies and therefore can be a first line, second line, third line, fourth line or fifth line therapy. The administration can be concurrently or simultaneous.

Methods to determine effectiveness of the therapy include reduction in tumor size or burden, prolonged progression-free survival, prolonged overall survival and reduced toxicity.

These methods for detecting efficacy can be combined with other clinical markers, e.g., a reduction in a tumor marker, e.g., CA125 or methods to determine the identity of the checkpoint expressed by the cancer cell or tumor. In a further aspect, a different cancer therapy or tumor resection is provided to the subject with the method.

In one aspect, administration comprises intravenous delivery.

In one embodiment, administration achieves one or more of: inhibiting metastatic potential of the cancer; reduction in tumor size; a reduction in tumor burden, longer progression free survival and longer overall survival of the subject.

In a further aspect, provided herein is a method for altering an immune cell profile of a cancer cell optionally expressing an immune checkpoint, e.g., PD-1. As is apparent to the skilled artisan, the peptide used for the method must correspond to the checkpoint expressed on the cell, e.g., an anti-PD-1 peptide for a cell expressing PD-1. The method comprises, or alternatively consists essentially of, or yet further consists of the nanoparticle, polynucleotide, vector, the composition and/or the host cell of this disclosure. In one aspect, an additional therapy is administered to the subject. The administration can be concurrently or simultaneous. When practiced in a non-human subject or a human subject, the method comprises administering an effective amount of the composition as described herein. In an animal model the method can be used to test for new drug combinations or for therapy itself. The treatment can be combined with other therapies and therefore can be a first line, second line, third line, fourth line or fifth line therapy. Methods to determine effectiveness of the therapy are known in the art and described herein.

In one particular aspect, the present disclosure provides kits for performing the methods of this disclosure as well as instructions for carrying out the methods of the present disclosure. The kit comprises, or alternatively consists essentially of, or yet further consists of one or more of the nanoparticle, polynucleotide, vector, the composition and/or the host cell of this disclosure. In one aspect, an additional therapy is provided in the kit.

In a further aspect, the instructions for use provide directions to conduct any of the methods disclosed herein.

### BRIEF DESCRIPTION OF THE DRAWINGS

FIGS. 1A-1B: Conjugation scheme. (FIG. 1A) Amino acid sequence of the anti-PD-1 peptide SNTSESF (SEQ ID NO: 1) and its linker and carboxy-terminal cysteine reside. (FIG. 1B) Bioconjugation scheme showing CPMV and its solvent exposed amine groups from lysine side chains followed by conjugation of the SM-(PEG)₈ linker introducing a maleimide group that then reacts with the cysteine side chain of the peptide

(SEQ ID NO: 2)
SNTSESFGSGGGSGGC.

FIGS. 2A-2D: Characterization of CPMV-AUNP. (FIG. 2A) Agarose gel electrophoresis of CPMV, CPMV-SMPEG, and CPMV-AUNP stained for GelRed (RNA detection) and Coomassie Blue (protein detection) and imaged under UV and white light, respectively. (FIG. 2B) SDS-PAGE analysis of the denatured coat proteins, S and L of CPMV, as well as the AUNP-conjugated versions thereof. S and L protein have a molecular weight of 24 kDa and 42 kDa, respectively and

$$SNTSESF \underline{GSGGGSGG} C \qquad ( SEQ ID \text{NO:} 2 )$$

has a molecular weight of 1.3 kDa. The left lane shows the molecular weight of the See Blue Plus 2 protein marker. (FIG. 2C) TEM of negatively stained CPMV and CPMV-AUNP. The scale bars are 100 nm and 50 nm in the insets. (FIG. 2D) SEC using a Superose-6 increase column on the ÄKTA Explorer system; RNA is monitored using a 260 nm and protein is monitored at 280 nm detector.

FIGS. 3A-3B: (FIG. 3A) Female C57BL/6 mice were inoculated (i.p.) with 2×10⁶ ID8-Defb29/Vegf-A cells followed by six weekly injections (i.p.) of PBS control (n=5), 1 μg AUNP (n=3), 100 μg CPMV (n=5), 100 μg CPMV-AUNP (n=7), 100 μg CPMV+1 μg AUNP physical mixture (n=7). Body weight was measured to monitor tumor growth. Data are means±SEM. Data are plotted for a minimum of n=3 per group. (FIG. 3B) Survival curves of the treatment groups.

### DETAILED DESCRIPTION

### Definitions

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the disclosure to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the disclosure. All publications, patent applications, patents and other references mentioned herein are incorporated by reference in their entirety.

Unless the context indicates otherwise, it is specifically intended that the various features of the disclosure described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

Throughout this disclosure, various publications, patents and published patent specifications are referenced by an identifying citation or by an Arabic numeral. The full citation for the publications identified by an Arabic numeral are found immediately preceding the claims. The disclosures of these publications, patents and published patent specifications are hereby incorporated by reference into the present disclosure in their entirety to more fully describe the state of the art to which this disclosure pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the description of the disclosure and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

As used herein, the term “comprising” is intended to mean that the compositions or methods include the recited steps or elements, but do not exclude others. “Consisting essentially of” shall mean rendering the claims open only for the inclusion of steps or elements, which do not materially affect the basic and novel characteristics of the claimed compositions and methods. “Consisting of” shall mean excluding any element or step not specified in the claim. Embodiments defined by each of these transition terms are within the scope of this disclosure

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

Cowpea chlorotic mottle virus (CCMV) is a spherical plant virus that belongs to the Bromovirus genus. Several strains have been identified and include, but not limited to, Carl (Ali, et al., 2007. J. Virological Methods 141:84-86), Car2 (Ali, et al., 2007. J. Virological Methods 141:84-86, 2007), type T (Kuhn, 1964. Phytopathology 54:1441-1442), soybean(S) (Kuhn, 1968. Phytopathology 58:1441-1442), mild (M) (Kuhn, 1979. Phytopathology 69:621-624), Arkansas (A) (Fulton, et al., 1975. Phytopathology 65:741-742), bean yellow stipple (BYS) (Fulton, et al., 1975. Phytopathology 65:741-742), R (Sinclair, ed. 1982. Compendium of Soybean Diseases. 2^{nd} ed. The American Physiopathological Society, St. Paul. 104 pp.), and PSM (Paguio, et al., 1988. Plant Diseases 72 (9): 768-770).

In some cases, the CCMV capsid comprise, or consists essentially of, or yet further consists of, s the sequence as set forth in the UniProtKB ID P03601:

(SEQ ID NO: 7)
MSTVGTGKLTRAQRRAAARKNKRNTRVVQPVIVEPIASGQGKAIKAWTG
YSVSKWTASCAAAEAKVTSAITISLPNELSSERNKQLKVGRVLLWLGLL
PSVSGTVKSCVTETQTTAAASFQVALAVADNSKDVVAAMYPEAFKGITL
EQLTADLTIYLYSSAALTEGDVIVHLEVEHVRPTFDDSFTPVY,

or an equivalent thereof.

In some cases, the CCMV is prepared by the method as described in Ali et al., “Rapid and efficient purification of Cowpea chlorotic mottle virus by sucrose cushion ultracentrifugation,” Journal of Virological Methods 141:84-86 (2007).

Cowpea mosaic virus (CPMV) is a non-enveloped plant virus that belongs to the Comovirus genus. CPMV strains include, but are not limited to, SB (Agrawal, H. O. (1964). Meded. Landb. Hoogesch. Wagen. 64:1) and Vu (Agrawal, H. O. (1964). Meded. Landb. Hoogesch. Wagen. 64:1).

In some instances, the CPMV comprise, or consists essentially of, or yet further consists of, a plurality of capsid proteins. In some instances, CPMV produces a large capsid protein and a small capsid protein precursor (which generates a mature small capsid protein). In some cases, CPMV capsid is formed from a plurality of large capsid proteins and mature small capsid proteins. In some cases, the large capsid protein is a wild-type large capsid protein, optionally expressed by SB or Vu strain. In other instances, the large capsid protein is a modified large capsid protein, e.g., comprising, or consisting essentially of, or yet further consisting of, one or more substitutions, insertions, and/or deletions. In some cases, the large capsid protein comprise, or consists essentially of, or yet further consists of, the sequence as set forth in the UniProtKB ID P03599 (residues 460-833):

(SEQ ID NO: 8)
MEQNLFALSLDDTSSVRGSLLDTKFAQTRVLLSKAMAGGDVLLDEYLYD
VVNGQDFRATVAFLRTHVITGKIKVTATTNISDNSGCCLMLAINSGVRG
KYSTDVYTICSQDSMTWNPGCKKNFSFTFNPNPCGDSWSAEMISRSRVR
MTVICVSGWTLSPTTDVIAKLDWSIVNEKCEPTIYHLADCQNWLPLNRW
MGKLTFPQGVTSEVRRMPLSIGGGAGATQAFLANMPNSWISMWRYFRGE
LHFEVTKMSSPYIKATVTFLIAFGNLSDAFGFYESFPHRIVQFAEVEEK
CTLVFSQQEFVTAWSTQVNPRTTLEADGCPYLYAIIHDSTTGTISGDFN
LGVKLVGIKDFCGIGSNPGIDGSRLLGAIAQ,

or an equivalent thereof.

As used herein, an immune checkpoint or checkpoint refers to a regulator and/or modulator of the immune system (such as an immune response, an anti-tumor immune response, a nascent anti-tumor immune response, an anti-tumor immune cell response, an anti-tumor T cell response, and/or an antigen recognition of T cell receptor in the process of immune response). Their interaction activates either inhibitory or activating immune signaling pathways. Thus a checkpoint may contain one of the two signals: a stimulatory immune checkpoint that stimulates an immune response, and an inhibitory immune checkpoint inhibiting an immune response. In some embodiments, the immune checkpoint is crucial for self-tolerance, which prevents the immune system from attacking cells indiscriminately. However, some cancers can protect themselves from attack by stimulating immune checkpoint targets. In some embodiments, the immune checkpoints are present on T cells, antigen-presenting cells (APCs) and/or tumor cells.

A checkpoint inhibitor is type of drug that blocks proteins called checkpoints that are made by some types of immune system cells, such as T cells, and some cancer cells. These checkpoints help keep immune responses from being too strong and sometimes can keep T cells from killing cancer cells. When these checkpoints are blocked, T cells can kill cancer cells better. Examples of checkpoint proteins found on T cells or cancer cells include PD-1/PD-L1 and CTLA-4/B7-1/B7-2. Some immune checkpoint inhibitors are used to treat cancer.

In some embodiments, the checkpoint inhibitor comprises, consists essentially of, or consists of a peptide that includes a protein or a fragment thereof from one or more selected from an anti-PD-1 agent, an anti-PD-L1 agent, an anti-CTLA-4 agent, an anti-LAG-3 agent, an anti-TIM-3 agent, an anti-TIGIT agent, an anti-VISTA agent, an anti-B7-H3 agent, an anti-BTLA agent, an anti-ICOS agent, an anti-GITR agent, an anti-4-1BB agent, an anti-OX40 agent, an anti-CD27 agent, an anti-CD28 agent, an anti-CD40 agent, and an anti-Siglec-15 agent. In some embodiments, the anti-PD-1 agent, the anti-PD-L1 agent, the anti-CTLA-4 agent, the anti-LAG-3 agent, the anti-TIM-3 agent, the anti-TIGIT agent, the anti-VISTA agent, the anti-B7-H3 agent, the anti-BTLA agent, the anti-ICOS agent, the anti-GITR agent, the anti-4-1BB agent, the anti-OX40 agent, the anti-CD27 agent, the anti-CD28 agent, the anti-CD40 agent, or the anti-Siglec-15 agent is an antagonist. In some embodiments, the anti-PD-1 agent, the anti-PD-L1 agent, the anti-CTLA-4 agent, the anti-LAG-3 agent, the anti-TIM-3 agent, the anti-TIGIT agent, the anti-VISTA agent, the anti-B7-H3 agent, the anti-BTLA agent, the anti-ICOS agent, the anti-GITR agent, the anti-4-1BB agent, the anti-OX40 agent, the anti-CD27 agent, the anti-CD28 agent, the anti-CD40 agent, or the anti-Siglec-15 agent is an agonist. In some embodiments, the anti-PD-1 agent, the anti-PD-L1 agent, the anti-CTLA-4 agent, the anti-LAG-3 agent, the anti-TIM-3 agent, the anti-TIGIT agent, the anti-VISTA agent, the anti-B7-H3 agent, the anti-BTLA agent, the anti-ICOS agent, the anti-GITR agent, the anti-4-1BB agent, the anti-OX40 agent, the anti-CD27 agent, the anti-CD28 agent, the anti-CD40 agent, or the anti-Siglec-15 agent is an inhibitor. In some embodiments, the anti-LAG-3 agent comprises, consists essentially of, or consists of AK104, KN046, eftilagimod alpha, relatlimab, LAG525, MK-4280, REGN3767, TSR-033, BI754111, Sym022, FS118, or MGD013. In some embodiments, the anti-TIM-3 agent comprises, consists essentially of, or consists of CA-327, TSR-022, MBG453, Sym023, INCAGN2390, LY3321367, BMS-986258, SHR-1702, or RO7121661. In some embodiments, the anti-TIGIT agent comprises, consists essentially of, or consists of MK-7684, etigilimab, tiragolumab, BMS-986207, AB-154, or ASP-8374. In some embodiments, the anti-VISTA agent comprises, consists essentially of, or consists of JNJ-61610588 or CA-170. In some embodiments, the anti-B7-H3 agent comprises, consists essentially of, or consists of enoblituzumab, MGD009, or omburtamab. In some embodiments, the anti-BTLA agent comprises, consists essentially of, or consists of TAB004/JS004. In some embodiments, the anti-Siglec-15 agent comprises, consists essentially of, or consists of NC318. In some embodiments, the checkpoint inhibitor comprises, consists essentially of, or consists of AK104 or KN046.

In some embodiments, the checkpoint inhibitor comprises, consists essentially of, or consists of a peptide or fragment thereof of an anti-PD1 agent or an anti-PD-L1 agent.

In some embodiments, the anti-PD1 agent comprises, consists essentially of, or consists of an anti-PD1 antibody or an antigen binding fragment thereof. In some embodiments, the anti-PD1 antibody comprises, consists essentially of, or consists of nivolumab, pembrolizumab, cemiplimab, spartalizumab, camrelizumab, sintilimab, tislelizumab, toripalimab, AMF 514 (MEDI0680), balstilimab, or a combination of two or more thereof.

In some embodiments, the anti-PD-L1 agent or fragment thereof comprises, consists essentially of, or consists of an anti-PD-L1 antibody or an antigen binding fragment thereof. In some embodiments, the anti-PD-L1 antibody comprises, consists essentially of, or consists of avelumab, durvalumab, atezolizumab, envafolimab, or a combination of two or more thereof.

In some embodiments, the checkpoint inhibitor comprises, consists essentially of, or consists of an anti-CTLA-4 agent or a fragment thereof. In some embodiments, the anti-CTLA-4 agent comprises, consists essentially of, or consists of an anti-CTLA-4 antibody or an antigen binding fragment thereof. In some embodiments, the anti-CTLA-4 antibody comprises, consists essentially of, or consists of ipilimumab, tremelimumab, zalifrelimab, or AGEN1181, or a combination thereof.

In some aspects, an additional therapy is contacted or administered to the subject. These can include adjuvant or neoadjuvant treatments. The term “adjuvant” therapy refers to administration of a therapy or chemotherapeutic regimen to a patient in addition to the primary or initial treatment, such as after removal of a tumor by surgery. Adjuvant therapy is typically given to minimize or prevent a possible cancer reoccurrence. Alternatively, “neoadjuvant” therapy refers to administration of therapy or chemotherapeutic regimen before surgery, typically in an attempt to shrink the tumor prior to a surgical procedure to minimize the extent of tissue removed during the procedure. Additionally or alternatively, such adjuvant therapy potentials (i.e., sensitizes the subject to the original therapy) the subject may help reach one or more of clinical end points of the cancer treatment.

Alternatively or additionally, the additional agents can include therapies to minimize side effects or to enhance the therapy or treatment such as chemotherapeutic agents, non-limiting example of such are provided herein. In one aspect, an additional therapy comprises 5-Fluorouracil (5-FU) which belongs to the family of therapy drugs called pyrimidine based anti-metabolites. It is a pyrimidine analog, which is transformed into different cytotoxic metabolites that are then incorporated into DNA and RNA thereby inducing cell cycle arrest and apoptosis. Chemical equivalents are pyrimidine analogs which result in disruption of DNA replication. Chemical equivalents inhibit cell cycle progression at S phase resulting in the disruption of cell cycle and consequently apoptosis. Equivalents to 5-FU include prodrugs, analogs and derivative thereof such as 5′-deoxy-5-fluorouridine (doxifluridine), 1-tetrahydrofuranyl-5-fluorouracil (ftorafur), capecitabine (Xeloda®), S-1 (MBMS-247616, consisting of tegafur and two modulators, a 5-chloro-2,4-dihydroxypyridine and potassium oxonate), raltitrexed (tomudex), nolatrexed (Thymitaq, AG337), LY231514 and ZD9331, as described for example in Papamichael (1999) The Oncologist 4:478-487.

A further example is “5-FU based adjuvant therapy” that refers to 5-FU alone or alternatively the combination of 5-FU with one or more other treatments, that include, but are not limited to radiation, methyl-CCNU, leucovorin, oxaliplatin (such as cisplatin), irinotecan, mitomycin, cytarabine, doxorubicin, cyclophosphamide, and levamisole, as well as an immunotherapy. Specific treatment adjuvant regimens are known in the art such as weekly Fluorouracil/Leucovorin, weekly Fluorouracil/Leucovorin+Bevacizumab, FOLFOX, FOLFOX-4, FOLFOX6, modified FOLFOX6 (mFOLFOX6), FOLFOX6 with bevacizumab, mFOLFOX6+Cetuximab, mFOLFOX6+Panitumumab, modified FOLFOX7 (mFOLFOX7), FOLFIRI, FOLFIRI with Bevacizumab, FOLFIRI+Ziv-aflibercept, FOLFIRI with Cetuximab, FOLFIRI+Panitumumab, FOLFIRI+Ramucirumab, FOLFOXIRI, FOLFIRI with FOLFOX6, FOLFOXIRI+Bevacizumab, FOLFOXIRI+Cetuximab, FOLFOXIRI+Panitumumab, Roswell Park Fluorouracil/Leucovorin, Roswell Park Fluorouracil/Leucovorin+Bevacizumab, Simplified Biweekly Infusional Fluorouracil/Leucovorin, Simplified Biweekly Infusional Fluorouracil/Leucovorin+Bevacizumab, and MOF (semustine(methyl-CCNU), vincrisine (Oncovin®) and 5-FU). For a review of these therapies see Beaven and Goldberg (2006) Oncology 20 (5): 461-470 as well as www.cancertherapyadvisor.com/home/cancer-topics/gastrointestinal-cancers/gastrointestinal-cancers-treatment-regimens/colon-cancer-treatment-regimens/. Other chemotherapeutics can be added, e.g., oxaliplatin or irinotecan.

Another example is capecitabine which is a prodrug of (5-FU) that is converted to its active form by the tumor-specific enzyme PynPase following a pathway of three enzymatic steps and two intermediary metabolites, 5′-deoxy-5-fluorocytidine (5′-DFCR) and 5′-deoxy-5-fluorouridine (5′-DFUR). Capecitabine is marketed by Roche under the trade name Xeloda®.

Leucovorin (Folinic acid) is another example. It is an adjuvant used in cancer therapy. It is used in synergistic combination with 5-FU to improve efficacy of the chemotherapeutic agent. Without being bound by theory, addition of Leucovorin is believed to enhance efficacy of 5-FU by inhibiting thymidylate synthase. It has been used as an antidote to protect normal cells from high doses of the anticancer drug methotrexate and to increase the antitumor effects of fluorouracil (5-FU) and tegafur-uracil. It is also known as citrovorum factor and Wellcovorin. This compound has the chemical designation of L-Glutamic acid N-[4-[[(2-amino-5-formyl-1,4,5,6,7,8-hexahydro-4-oxo-6-pteridinyl)methyl]amino]benzoyl], calcium salt (1:1).

Another example is “oxaliplatin” (Eloxatin) which is a platinum-based chemotherapy drug in the same family as cisplatin and carboplatin. It is typically administered in combination with fluorouracil and leucovorin in a combination known as FOLFOX for the treatment of colorectal cancer. Compared to cisplatin, the two amine groups are replaced by cyclohexyldiamine for improved antitumor activity. The chlorine ligands are replaced by the oxalato bidentate derived from oxalic acid in order to improve water solubility. Equivalents to Oxaliplatin are known in the art and include, but are not limited to cisplatin, carboplatin, aroplatin, lobaplatin, nedaplatin, and JM-216 (see Mckeage et al. (1997) J. Clin. Oncol. 201:1232-1237 and in general, Chemotherapy for Gynecological Neoplasm, Curr. Therapy and Novel Approaches, in the Series Basic and Clinical Oncology, Angioli et al. Eds., 2004).

A further example of an additional therapy is “FOLFOX” which is an abbreviation for a type of combination therapy that is used to treat cancer. This therapy includes leucovorin (“FOL”), 5-FU (“F”), and oxaliplatin (“OX”) and encompasses various regimens, such as FOLFOX-4, FOLFOX-6, modified FOLOX-6, and FOLFOX-7, which vary in doses and ways in which each of the three drugs are administered. “FOLFIRI” is an abbreviation for a type of combination therapy that is used treat cancer and comprises, or alternatively consists essentially of, or yet further consists of 5-FU, leucovorin, and irinotecan. Information regarding these treatments are available on the National Cancer Institute's web site, cancer.gov, last accessed on May 30, 2020 as well as www.cancertherapyadvisor.com/home/cancer-topics/gastrointestinal-cancers/gastrointestinal-cancers-treatment-regimens/colon-cancer-treatment-regimens/, last accessed on May 30, 2020.

A further example includes Irinotecan (CPT-11) which is sold under the trade name of Camptosar. It is a semi-synthetic analogue of the alkaloid camptothecin, which is activated by hydrolysis to SN-38 and targets topoisomerase I. Chemical equivalents are those that inhibit the interaction of topoisomerase I and DNA to form a catalytically active topoisomerase I-DNA complex. Chemical equivalents inhibit cell cycle progression at G2-M phase resulting in the disruption of cell proliferation.

Yet further examples include biologics such as monoclonal antibodies and therapies derived from such.

The term “adjuvant” therapy refers to administration of a therapy or chemotherapeutic regimen to a patient in addition to the primary or initial treatment, such as after removal of a tumor by surgery. Adjuvant therapy is typically given to minimize or prevent a possible cancer reoccurrence. Alternatively, “neoadjuvant” therapy refers to administration of therapy or chemotherapeutic regimen before surgery, typically in an attempt to shrink the tumor prior to a surgical procedure to minimize the extent of tissue removed during the procedure. Additionally or alternatively, such adjuvant therapy potentials (i.e., sensitizes the subject to the original therapy) the subject may help reach one or more of clinical end points of the cancer treatment.

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder.

“Eukaryotic cells” comprise, or alternatively consist essentially of, or yet further consist of all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human,

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

A “composition” typically intends a combination of the active agent, e.g., the nanoparticle of this disclosure and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, including cells, treatments, therapies, agents, drugs and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “isolated cell” generally refers to a cell that is substantially separated from other cells of a tissue. The term includes prokaryotic and eukaryotic cells.

As used herein, the phrase “immune response” or its equivalent “immunological response” refers to the development of a cell-mediated response (e.g. mediated by antigen-specific T cells or their secretion products). A cellular immune response is elicited by the presentation of polypeptide epitopes in association with Class I or Class II MHC molecules, to treat or prevent a viral infection, expand antigen-specific B-reg cells, TC1, CD4+ T helper cells and/or CD8+ cytotoxic T cells and/or disease generated, autoregulatory T cell and B cell “memory” cells. The response may also involve activation of other components. In some aspect, the term “immune response” may be used to encompass the formation of a regulatory network of immune cells. Thus, the term “regulatory network formation” may refer to an immune response elicited such that an immune cell, preferably a T cell, more preferably a T regulatory cell, triggers further differentiation of other immune cells, such as but not limited to, B cells or antigen-presenting cells-non-limiting examples of which include dendritic cells, monocytes, and macrophages. In certain embodiments, regulatory network formation involves B cells being differentiated into regulatory B cells; in certain embodiments, regulatory network formation involves the formation of tolerogenic antigen-presenting cells.

The term “immune cells” includes, e.g., white blood cells (leukocytes) which are derived from hematopoietic stem cells (HSC) produced in the bone marrow, lymphocytes (T cells, B cells, natural killer (NK) cells) and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Cytokines are small secreted proteins released by immune cells that have a specific effect on the interactions and communications between the immune cells. Cytokines can be pro-inflammatory or anti-inflammatory. Non-limiting example of a cytokine is Granulocyte-macrophage colony-stimulating factor (GM-CSF), which stimulates stem cells to produce granulocytes (neutrophils, eosinophils, and basophils) and monocytes.

As used herein, the term “vector” refers to a nucleic acid construct deigned for transfer between different hosts, including but not limited to a plasmid, a virus, a cosmid, a phage, a BAC, a YAC, etc. A “viral vector” is defined as a recombinantly produced virus or viral particle that comprises a polynucleotide to be delivered into a host cell, either in vivo, ex vivo or in vitro. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Further details as to modern methods of vectors for use in gene transfer may be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif.) and Promega Biotech (Madison, Wis.).

An “effective amount” or “efficacious amount” refers to the amount of an agent or combined amounts of two or more agents, that, when administered for the treatment of a mammal or other subject, is sufficient to effect such treatment for the disease. The “effective amount” will vary depending on the agent(s), the disease and its severity and the age, weight, etc., of the subject to be treated.

In some embodiments the effective amount will depend on the size and nature of the application in question. It will also depend on the nature and sensitivity of the target subject and the methods in use. The skilled artisan will be able to determine the effective amount based on these and other considerations. The effective amount may comprise, or alternatively consist essentially of, or yet further consist of one or more administrations of a composition depending on the embodiment.

In one embodiment, the term “disease” or “disorder” as used herein refers to a cancer or a tumor (which are used interchangeably herein), a status of being diagnosed with such disease, a status of being suspect of having such disease, or a status of at high risk of having such disease.

As used herein, “cancer” or “malignancy” or “tumor” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features.

When the disease is cancer, the following clinical endpoints are non-limiting examples of treatment: (1) elimination of a cancer in a subject or in a tissue/organ of the subject or in a cancer loci; (2) reduction in tumor burden (such as number of cancer cells, number of cancer foci, number of cancer cells in a foci, size of a solid cancer, concentrate of a liquid cancer in the body fluid, and/or amount of cancer in the body); (3) stabilizing or delay or slowing or inhibition of cancer growth and/or development, including but not limited to, cancer cell growth and/or division, size growth of a solid tumor or a cancer loci, cancer progression, and/or metastasis (such as time to form a new metastasis, number of total metastases, size of a metastasis, as well as variety of the tissues/organs to house metastatic cells); (4) less risk of having a cancer growth and/or development; (5) inducing an immune response of the patient to the cancer, such as higher number of tumor-infiltrating immune cell, higher number of activated immune cells, or higher number cancer cell expressing an immunotherapy target, or higher level of expression of an immunotherapy target in a cancer cell; (6) higher probability of survival and/or increased duration of survival, such as increased overall survival (OS, which may be shown as 1-year, 2-year, 5-year, 10-year, or 20-year survival rate), increased progression free survival (PFS), increased disease free survival (DFS), increased time to tumor recurrence (TTR) and increased time to tumor progression (TTP). In some embodiments, the subject after treatment experiences one or more endpoints selected from tumor response, reduction in tumor size, reduction in tumor burden, increase in overall survival, increase in progression free survival, inhibiting metastasis, improvement of quality of life, minimization of drug-related toxicity, and avoidance of side-effects (e.g., decreased treatment emergent adverse events). In some embodiments, improvement of quality of life includes resolution or improvement of cancer-specific symptoms, such as but not limited to fatigue, pain, nausea/vomiting, lack of appetite, and constipation; improvement or maintenance of psychological well-being (e.g., degree of irritability, depression, memory loss, tension, and anxiety); improvement or maintenance of social well-being (e.g., decreased requirement for assistance with eating, dressing, or using the restroom; improvement or maintenance of ability to perform normal leisure activities, hobbies, or social activities; improvement or maintenance of relationships with family). In some embodiments, improved patient quality of life that is measured qualitatively through patient narratives or quantitatively using validated quality of life tools known to those skilled in the art, or a combination thereof. Additional non-limiting examples of endpoints include reduced hospital admissions, reduced drug use to treat side effects, longer periods off-treatment, and earlier return to work or caring responsibilities. In one aspect, prevention or prophylaxis is excluded from treatment.

In certain embodiments, the terms “disease” “disorder” and “condition” are used interchangeably herein, referring to a cancer, a status of being diagnosed with a cancer, or a status of being suspect of having a cancer. “Cancer”, which is also referred to herein as “tumor”, is a known medically as an uncontrolled division of abnormal cells in a part of the body, benign or malignant. In one embodiment, cancer refers to a malignant neoplasm, a broad group of diseases involving unregulated cell division and growth, and invasion to nearby parts of the body. Non-limiting examples of cancers include carcinomas, sarcomas, leukemia and lymphoma, e.g., colon cancer, ovarian cancer, colorectal cancer, rectal cancer, gastric cancer, esophageal cancer, head and neck cancer, breast cancer, brain cancer, lung cancer, stomach cancer, liver cancer, gall bladder cancer, or pancreatic cancer. In one embodiment, the term “cancer” refers to a solid tumor, which is an abnormal mass of tissue that usually does not contain cysts or liquid areas, including but not limited to, sarcomas, carcinomas, and certain lymphomas (such as Non-Hodgkin's lymphoma). In another embodiment, the term “cancer” refers to a liquid cancer, which is a cancer presenting in body fluids (such as, the blood and bone marrow), for example, leukemias (cancers of the blood) and certain lymphomas.

A “solid tumor” is an abnormal mass of tissue that usually does not contain cysts or liquid areas. Solid tumors can be benign or malignant. Different types of solid tumors are named for the type of cells that form them. Examples of solid tumors include, but not limited to, sarcomas, carcinomas, and lymphomas. In some embodiments, a solid tumor comprises bladder cancer, bone cancer, ovarian cancer, brain cancer, breast cancer, colorectal cancer, esophageal cancer, eye cancer, head and neck cancer, kidney cancer, lung cancer, melanoma, ovarian cancer, pancreatic cancer, prostate cancer, skin cancer, gastric cancer, esophageal cancer, colon cancer, glioma, cervical cancer, hepatocellular, thyroid cancer, or stomach cancer.

Additionally or alternatively, a cancer may refer to a local cancer (which is an invasive malignant cancer confined entirely to the organ or tissue where the cancer began), a metastatic cancer (referring to a cancer that spreads from its site of origin to another part of the body), a non-metastatic cancer, a primary cancer (a term used describing an initial cancer a subject experiences), a secondary cancer (referring to a metastasis from primary cancer or second cancer unrelated to the original cancer), an advanced cancer, an unresectable cancer, or a recurrent cancer. As used herein, an advanced cancer refers to a cancer that had progressed after receiving one or more of: the first line therapy, the second line therapy, or the third line therapy.

As used herein, a “cancer cell” are cells that have uncontrolled cell division and form solid tumors or enter the blood stream.

As used herein, the term “administer” or “administration” or “administering” intends to mean delivery of a substance to a subject such as an animal or human. Administration can be effected in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and animals, treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and the target cell or tissue. Non-limiting examples of route of administration include intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmucosal, and inhalation.

An agent of the present disclosure can be administered for therapy by any suitable route of administration. It will also be appreciated that the optimal route will vary with the condition and age of the recipient, and the disease being treated.

“Therapeutically effective amount” of a drug or an agent refers to an amount of the drug or the agent that is an amount sufficient to obtain a pharmacological response such as passive immunity; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding the chimeric PVX described herein). Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical,” percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

It is to be inferred without explicit recitation and unless otherwise intended, that when the present disclosure relates to a polypeptide, protein, polynucleotide, an equivalent or a biologically equivalent of such is intended within the scope of this disclosure. As used herein, the term “biological equivalent thereof” is intended to be synonymous with “equivalent thereof” when referring to a reference protein, polypeptide or nucleic acid, intends those having minimal homology while still maintaining desired structure or functionality. Unless specifically recited herein, it is contemplated that any of the above also includes equivalents thereof. For example, an equivalent intends at least about 70% homology or identity, or at least 80% homology or identity and alternatively, or at least about 85%, or alternatively at least about 90%, or alternatively at least about 95%, or alternatively at least 98% percent homology or identity and/or exhibits substantially equivalent biological activity to the reference protein, polypeptide, or nucleic acid. Alternatively, when referring to polynucleotides, an equivalent thereof is a polynucleotide that hybridizes under stringent conditions to the reference polynucleotide or its complement.

The phrase “equivalent polypeptide” or “equivalent peptide fragment” refers to protein, polynucleotide, or peptide fragment encoded by a polynucleotide that hybridizes to a polynucleotide encoding the exemplified polypeptide or its complement of the polynucleotide encoding the exemplified polypeptide, under high stringency and/or which exhibit similar biological activity in vivo, e.g., approximately 100%, or alternatively, over 90% or alternatively over 85% or alternatively over 70%, as compared to the standard or control biological activity. Additional embodiments within the scope of this disclosure are identified by having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence homology. Percentage homology can be determined by sequence comparison using programs such as BLAST run under appropriate conditions. In one aspect, the program is run under default parameters.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. A high stringency hybridization refers to a condition in which hybridization of an oligonucleotide to a target sequence comprises no mismatches (or perfect complementarity). Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

The term “PEG” as used herein refers to polyethylene glycol, a polyether compound derived from petroleum with many applications, from industrial manufacturing to medicine. PEG is also known as polyethylene oxide (PEO) or polyoxyethylene (POE), depending on its molecular weight. The structure of PEG is commonly expressed as H—(O—CH2—CH2)ₙ—OH.

The term “click chemistry” as used herein refers to a class of biocompatible small molecule reactions commonly used for tasks such as bioconjugation. In one non-limiting example, click chemistry is used to join a reporter molecule to a biomolecule.

Click chemistry reactions are not a single reaction, but rather a group of reactions with similar characteristics. Click chemistry reactions have several distinctive characteristics such as they usually occur in one vessel, are not disturbed by water, generate minimal byproducts, and have a high thermodynamic driving force. One non-limiting example of such a reaction is the copper (I) catalyzed azide-alkyne cycloaddition reaction to form a 5-membered heteroatom ring. When the azide is located on one molecule, and the alkyne on another molecule, this reaction joins the two molecules in a covalent manner.

“Lyophilized” refers the process of lyophilization, which involves removing water from a product after it is frozen and placed under a vacuum, allowing the ice to change directly from solid to vapor without passing through a liquid phase.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

As used herein, the term “overexpress” with respect to a cell, a tissue, or an organ expresses a protein to an amount that is greater than the amount that is produced in a control cell, a control issue, or an organ. A protein that is overexpressed may be endogenous to the host cell or exogenous to the host cell.

As used herein, the term “enhancer”, denotes sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

The term “introduce” as applied to methods of producing modified cells such as chimeric antigen receptor cells refers to the process whereby a foreign (i.e. extrinsic or extracellular) agent is introduced into a host cell thereby producing a cell comprising the foreign agent. Methods of introducing nucleic acids include but are not limited to transduction, retroviral gene transfer, transfection, electroporation, transformation, viral infection, and other recombinant DNA techniques known in the art. In some embodiments, transduction is done via a vector (e.g., a viral vector). In some embodiments, transfection is done via a chemical carrier, DNA/liposome complex, or micelle (e.g., Lipofectamine (Invitrogen)). In some embodiments, viral infection is done via infecting the cells with a viral particle comprising the polynucleotide of interest (e.g., AAV). In some embodiments, introduction further comprises CRISPR mediated gene editing or Transcription activator-like effector nuclease (TALEN) mediated gene editing. Methods of introducing non-nucleic acid foreign agents (e.g., soluble factors, cytokines, proteins, peptides, enzymes, growth factors, signaling molecules, small molecule inhibitors) include but are not limited to culturing the cells in the presence of the foreign agent, contacting the cells with the agent, contacting the cells with a composition comprising the agent and an excipient, and contacting the cells with vesicles or viral particles comprising the agent.

The term “culturing” refers to growing cells in a culture medium under conditions that favor expansion and proliferation of the cell. The term “culture medium” or “medium” is recognized in the art and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for culture is a medium. Similarly, a powder mixture that when mixed with water or other liquid becomes suitable for cell culture may be termed a “powdered medium.” “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high-density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. In one aspect, the growth medium may be a complex medium with the necessary growth factors to support the growth and expansion of the cells of the disclosure while maintaining their self-renewal capability. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, McCoy's 5A, Dulbecco's MEM/F—I 2, RPMI 1640, and Iscove's Modified Dulbecco's Medium (IMDM).

### MODES FOR CARRYING OUT THE DISCLOSURE

Several in situ vaccine approaches are in pre-clinical and clinical development and therapies such as Imlygic (Amgen) are already in clinical use. Imlygic is an engineered oncolytic virus administered intralesional; tumor cell killing is achieved by the oncolytic function of the virus which is engineered to express the cytokine granulocyte-macrophage colony-stimulating factor (GM-CSF) to recruit and active innate immune cells to process TAAs and lead to systemic anti-tumor immunity [6]. As an alternative to the mammalian viruses, Applicant has focused on the development and study of plant viral immunotherapies. Applicant demonstrated the immunomodulatory properties of the plant virus cowpea mosaic virus (CPMV).

CPMV is an icosahedral plant virus measuring 30 nm in diameter; the virus particles are non-enveloped and non-glycosylated. Although CPMV is non-infectious toward mammals, it is immunogenic. The repetitive, multivalent protein assemblies are pathogen-associated molecular patterns (PAMPs) that act as danger signals and activate the innate immune system. The primary PAMP receptors that recognize proteins are Toll-like receptors (TLRs). Applicant's data indicate that RNA-free, empty CPMV (eCPMV) particles are recognized by TLR2 and TLR4. RNA-containing CPMV particles signal additionally through TLR7 [7]. In particular, CPMV signals through interferon gamma (IFN-γ) [8, 9] as well as type-I interferons [7]. The innate immune-stimulation recruits innate immune cells into the TME; i.e. reprogramming of M2 to M1 macrophages, infiltration of N1 neutrophils, etc. Tumor cell kill is initially mediated by neutrophils and Natural Killer (NK) cells; activation and recruitment of antigen presenting cells then leads to priming of systemic anti-tumor immunity and Applicant demonstrates priming of tumor-specific CD4⁺ and CD8⁺ cells including memory cells; the adaptive arm targets metastatic disease and induces immune memory [10]. Applicant has demonstrated that CPMV in situ vaccination stimulates a potent antitumor immune response in mouse models of melanoma, ovarian cancer, breast cancer, colon cancer [8, 9], and glioma [11]. CPMV induces systemic and durable immune-mediated anti-tumor efficacy accompanied with immunological memory to prevent recurrence [9]. Ongoing trials with melanoma indicate that the potent antitumor efficacy of CPMV can be replicated in these patients [12-14].

CPMV in situ vaccination primes innate immune cell activation, which leads to adaptive immune system-mediated, anti-tumor responses. These responses included increased tumor infiltration by CD4⁺ and CD8⁺ effector T cells and memory T cells. Following CPMV in situ vaccination, expression of PD-1 and PD-L1 is differentially increased in tumor models of melanoma, ovarian carcinoma, and colon carcinoma. Therefore, the CPMV treatment sensitizes the tumor to a specific immune checkpoint therapy and combination therapy showed dramatic increases in efficacy against tumors such as ovarian cancer [15].

Building on this prior work, Applicant set out to develop a next-generation CPMV displaying anti-PD-1 peptides. Small molecule agents and peptides have been developed to target the PD-1/PD-L1 axis. For example, the macrocyclic peptide BMS-986189 is undergoing clinical testing in a phase I clinical trial (NCT02739373). Another candidate is the D-peptide ^{D}PPA-1 (nyskptdrqyhf/SEQ ID NO: 3) which blocks the PD-1-PD-L1 interactions. The D configurations confers stability and in vivo efficacy was demonstrated in tumor mouse models [16]. Lastly, AUNP-12 a branched peptide with SNTSESF (SEQ ID NO: 1)-branched off the main sequence SNTSESFKFRVTQLAPKAQIKE (SEQ ID NO: 4) at the K residue (underlined). The peptide was designed to mimic the endogenous PD-1 receptor and inhibits PD-1 function; in particular, the side branch was shown to have surprisingly high activities [17]. Applicant used the side branch SNTSESF (SEQ ID NO: 1) and conjugated it to CPMV nanoparticles to show that CPMV displaying the anti-PD-1 peptide would show enhanced efficacy as in situ vaccine. Applicant reports herein the bioconjugation of CPMV-SNTSESF, referred to as CPMV-AUNP and demonstrate efficacy against cancer such as metastatic ovarian cancer.

### Embodiments

In some embodiments, a nanoparticle comprises, or alternatively consists essentially of, or yet further consists of a CPMV or a cowpea chlorotic mottle virus (CCMV) and a peptide that binds to an immune checkpoint, such as PD-1. In some embodiments, a nanoparticle comprises the amino acid of SEQ ID NO: 1 or 2, or an equivalent thereof at least 70% identical or similar to SEQ ID NO: 1 or 2. In other embodiments, the peptide comprises, or consist essentially of, or yet consists of, the D-peptide ^{D}PPA-1 (nyskptdrqyhf) (SEQ ID NO: 3) or an equivalent thereof having at least 70% identical, or at least 80%, or at least 90% or at least 95% sequence identity while still binding PD-1 and which blocks the PD-1-PD-L1 interactions. The D configurations confers stability and in vivo efficacy was demonstrated in tumor mouse models. In another aspect, the peptide comprises, or consists essentially of, or consists of, the branched peptide with SNTSESF (SEQ ID NO: 1)-branched off the main sequence SNTSESFKFRVTQLAPKAQIKE (SEQ ID NO: 4) at the K residue (underlined) or an equivalent thereof having at least 70% identical, or at least 80%, or at least 90% or at least 95% sequence identity while still binding PD-1. The peptide was designed to mimic the endogenous PD-1 receptor and inhibits PD-1 function. In some aspects, the peptide further comprises a linker GSGGGSGG (SEQ ID NO: 5) with an optional C-terminal cysteine (SEQ ID NO: 6). In another aspect, the peptide further comprises the linker with the C-terminal cysteine.

The number of peptides joined to the CPMV or CCMV can be 1 or more, or 2 or more or 5 or more, or 10 or more, or 15 or more, or 20 or more, or 25 or more, or 30 or more, or 35 or more, or 50 or more, or 100 or more, or 150 or more, or 200 or more, and ranges in between.

In some embodiments, the CMPV or CCMV has an exposed lysine side chain. In some embodiments, the peptide can be chemically conjugated or genetically fused to the CMPV or CCMV. Non-limiting examples of chemical conjugation include conjugating a thiol-terminated peptide through a maleimide-PEG-NHS linker targeting lysine groups on CMPV or CCMV. Azide/alkyne modified peptides and CMPV or CCMV and click chemistry can also be used for chemical conjugation. Any bioconjugation method would be applicable. For genetic fusion, the peptide is added as N-terminal fusion in a CMPV or CCMV plasmid containing the entire CMPV or CCMV genome. In some embodiments, a lysine side chain is conjugated to an N-hydroxysuccinimide (NHS) ester and the maleimide of a maleimide-polyethylene glycols is conjugated with the c-terminal cysteine of the peptide.

In some embodiments, the diameter of the nanoparticle disclosed herein, is from about 10 nm to 50 nm. In some embodiments, the diameter may range from about 10 nm, about 15 nm, about 20 nm, about 25 nm, about 30 nm, about 35 nm, about 40 nm, about 45 nm, to about 50 nm. The number of peptides joined to the CPMV or CCMV can be 1 or more, or 2 or more or 5 or more, or 10 or more, or 15 or more, or 20 or more, or 25 or more, or 30 or more, or 35 or more, or 50 or more, or 100 or more, or 150 or more, or 200 or more, and ranges in between.

Further provided herein, are a plurality of nanoparticles, where the nanoparticles are the same or different from each other. For example, the plurality can comprise the same or different peptide, diameter or linkers, as desired.

In some embodiments, a polynucleotide encodes a nanoparticle as disclosed herein. The polynucleotide can be RNA or DNA. In some embodiments, a vector as disclosed herein, comprises, or alternatively consists essentially of, or yet further consists of a nanoparticle as disclosed herein. In some embodiments, a host cell as disclosed herein, comprises, or alternatively consists essentially of, or yet further consists of a nanoparticle as disclosed herein. In one aspect, the vector is a plasmid. In one aspect, the host cell is a prokaryotic cell. In another aspect, the host cell is a eukaryotic cell. In one particular aspect, the host cell is a plant cell or a bacterium.

In some embodiments, a composition comprising, or alternatively consisting essentially of, or yet further consisting of the nanoparticle, polynucleotide, vector and/or host cell as disclosed herein. In another aspect, provided herein is a composition comprising, or alternatively consisting essentially of, or yet further consisting of a carrier and one or more of the nanoparticle, polynucleotide, vector and/or the host cell of this disclosure. In one aspect, the composition is lyophilized for ease of storage. The compositions can further comprise an additional therapeutic or other agent as known in the art or as described herein.

Further disclosed herein are methods for inducing an immune response in a subject consisting essentially of, or yet further consisting of the nanoparticles, polynucleotides, vectors and/or host cells as disclosed herein.

Further disclosed herein are methods for inhibiting the growth of a cancer cell optionally expressing a checkpoint comprising, or alternatively consisting essentially of, or yet further consisting of contacting the cells with the nanoparticles, polynucleotides, vectors and/or host cells as disclosed herein. In some embodiments, contacting is in vitro or in vivo. In one aspect, the immune checkpoint inhibitor is PD-1 and the peptide binds to PD-1 or PD-L1. As is apparent to the skilled artisan, the peptide on the nanoparticle targets the checkpoint of interest.

Further disclosed herein are methods for treating cancer optionally expressing an immune checkpoint in a subject in need thereof, comprising, or alternatively consisting essentially of, or yet further consisting of administering to the subject the nanoparticles, polynucleotides, vectors and/or host cells as disclosed herein. In one aspect, the checkpoint is PD-1 and the peptide binds to PD-1 or PD-L1. As is apparent to the skilled artisan, the peptide on the nanoparticle targets the immune checkpoint of interest.

Further disclosed herein are methods for altering an immune cell profile in a subject having a cancer or tumor optionally expressing a checkpoint, comprising, or alternatively consisting essentially of, or yet further consisting of nanoparticles, polynucleotides, vectors and/or host cells as disclosed herein. In one aspect, the checkpoint is PD-1 and the peptide binds to PD-1 or PD-L1. As is apparent to the skilled artisan, the peptide on the nanoparticle targets the immune checkpoint of interest.

In some embodiments, a subject is a mammal. In some embodiments, a subject is a human. In some embodiments, a subject has a condition. In some embodiments, a subject has cancer. In some embodiments, a cancer is selected from melanoma, breast cancer, prostate cancer, lung cancer, ovarian cancer, skin cancer, bladder cancer, pancreatic cancer, gastric cancer, esophageal cancer, colon cancer, glioma, cervical cancer, hepatocellular cancer, or thyroid cancer. In some embodiments, the cancer is primary or metastatic cancer. In some embodiments, the cancer is metastatic or primary ovarian cancer. In some embodiments, the cancer metastatic melanoma or metastatic triple negative breast cancer. In some embodiments, the cancer is a primary or metastatic ovarian cancer.

In some embodiments, administering is selected from intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmucosal, or inhalation. In some embodiments, administering is intravenous.

The methods and compositions disclosed herein may further comprise or alternatively consist essentially of, or yet further consists of administering to the subject an anti-tumor or other therapy to the benefit of the subject other than the nanoparticle disclosed herein. In some embodiments, anti-tumor therapy may include different cancer therapy or tumor resection.

In some embodiments, the nanoparticle and/or composition are provided to prevent the symptoms of cancer from occurring in a subject that is predisposed or does not yet display symptoms of the cancer.

In some embodiments, the polynucleotide, nanoparticle, vector, or composition disclosed herein may be delivered or administered into a cavity formed by the resection of tumor tissue (i.e. intracavity delivery) or directly into a tumor prior to resection (i.e. intratumoral delivery). In some embodiments, the administering is intravenous.

In some embodiments, any of the polynucleotides, nanoparticles, vectors, or compositions disclosed herein, are administered to the subject at least 1, 2, 3, 4, 5, 6, 7, 8, 9, or 10 times a day. In some embodiments, any of polynucleotides, nanoparticles, vectors, or compositions disclosed herein are administered to the subject at least 1, 2, 3, 4, 5, 6, 7, 8, 9, 10, 11, 12, 13, 14, 15, 16, 17, 18, 19, or 20 times a week. In some embodiments, any of the polynucleotides, nanoparticles, vectors, or compositions disclosed herein are administered to the subject at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 times a month. In some embodiments, any of the polynucleotides, nanoparticles, vectors, or compositions disclosed herein are administered to the subject at least every 1, 2, 3, 4, 5, 6, 7, 8, 9, or 10 days. In some embodiments, any of the polynucleotides, nanoparticles, vectors, or compositions disclosed herein are administered to the subject at least every 1, 2, 3, 4, 5, 6, 7, 8, 9, 10, 11, 12, 13, or 14 weeks. In some embodiments, any of the polynucleotides, nanoparticles, vectors, or compositions disclosed herein are administered to the subject for a period of at least 1, 2, 3, 4, 5, 6, 7, 8, 9, or 10 days. In some embodiments, any of the polynucleotides, nanoparticles, vectors, or compositions disclosed herein are administered to the subject for a period of at least 1, 2, 3, 4, 5, 6, 7, 8, 9, 10, 11, 12, 13, 14, 15, 16, 17, 18, 19, or 20 weeks. In some embodiments, any of the polynucleotides, nanoparticles, vectors, or compositions disclosed herein are administered to the subject for a period of at least 1, 2, 3, 4, 5, 6, 7, 8, 9, 10, 12, 13, 14, 15, 16, 17, 18, 19, or 20 months.

In some embodiments, the method and compositions provided herein, comprising, or alternatively consisting essentially of, or yet further consisting inhibiting metastatic potential of the cancer, reduction in tumor size, a reduction in tumor burden, longer progression free survival, or longer overall survival of the subject.

In one particular aspect, the present disclosure provides kits for performing the methods of this disclosure as well as instructions for carrying out the methods of the present disclosure. The kit comprises, or alternatively consists essentially of, or yet further consists of one or more of nanoparticle, polynucleotide, vector and/or host cell of this disclosure and instructions for use. In a further aspect, an additional therapy is provided in the kit. In a further aspect, the instruction for use provide directions to conduct any of the methods disclosed herein.

The kits are useful for detecting the presence of cancer such as ovarian cancer in a biological sample e.g., any bodily fluid including, but not limited to, e.g., sputum, serum, plasma, lymph, cystic fluid, urine, stool, cerebrospinal fluid, acidic fluid or blood and including biopsy samples of body tissue. The test samples may also be a tumor cell, a normal cell adjacent to a tumor, a normal cell corresponding to the tumor tissue type, a blood cell, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kit components, (e.g., reagents) can be packaged in a suitable container. The kit can also comprise, or alternatively consist essentially of, or yet further consist of, e.g., a buffering agent, a preservative or a protein-stabilizing agent. The kit can further comprise, or alternatively consist essentially of, or yet further consist of components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

As is apparent to those of skill in the art, the aforementioned methods and compositions can be combined with other therapeutic composition and agents for the treatment or the disclosed diseases or conditions.

### Materials and Methods

Production of Cowpea mosaic virus (CPMV): CPMV was propagated in Vigna unguiculata plants (Burpee's Black-eyed pea No. 5) and purified from infected leaves using previously described methods [34]. CPMV preparations were stored in 0.1 M potassium phosphate (KP) buffer pH 7.0 at proteins concentrations <10 mg/mL and 4° C. CPMV concentration was determined by UV/vis spectroscopy using a Nanodrop instrument and the CPMV specific extinction coefficient 8260 nm=8.1 mg⁻¹ mL cm⁻¹. The 260:280 nm ratio was also determined and intact CPMV has a 260:280 nm ratio of 1.8.

Synthesis of CPMV-AUNP: CPMV-AUNP was obtained by conjugating AUR-7 peptide [17],

(SEQ ID NO: 2)
SNTSESFGSGGGSGGC.

to CPMV using a two-step bioconjugation protocol.

(SEQ ID NO: 2)
SNTSESFGSGGGSGGC.

was obtained from Genscript with SNTSESF (SEQ ID NO: 1) being the active region with reported antagonist activity of the PD-1 pathway [17];

(SEQ ID NO: 5)
GSGGGSGG

was added as an intervening linker and the carboxy-terminal cysteine residue acts as ligation handle for conjugation to CPMV. First, CPMV was functionalized using a bi-functional N-hydroxysuccinimide-PEG₈-maleimide (SM-PEG₈) linker (Thermo Fisher Scientific). CPMV in 0.1 M KP buffer pH 7.4 was reacted with 500 M excess of SM-PEG₈ linker at room temperature with constant mixing for 2 h at a 1 mg/mL. Second,

(SEQ ID NO: 2)
SNTSESFGSGGGSGGC.

was added at 500, 1000 or 2000 M excess to CPMV. The resulting CPMV-AUNP conjugate was purified using 100 kDa molecular weight cut-off Amicon spin filters (Millipore). The product was resuspended in 0.1 M KP buffer pH 7.0 and stored at 4° C.

Native and denaturing gel electrophoresis: CPMV, CPMV-SMPEG and CPMV-AUNP (10 μg per lane) were analyzed using 1% (w/v) agarose gel electrophoresis in 0.1 M TAE buffer (pH 6.5). Gels were stained with GelRed to stain the encapsulated RNA and Coomassie Brilliant Blue to stain the protein capsid. Denatured protein subunits (˜10 μg per lane) were analyzed by polyacrylamide gel electrophoresis using 4-12% NuPAGE gels in 1×MOPS buffer (Invitrogen). Samples were denatured by boiling in SDS loading dye (Invitrogen) for 10 min. Gels were photographed under UV (when stained with GelRed) or white light (when stained with Coomassie Brilliant Blue) using an AlphaImager system (ProteinSimple).

Transmission electron microscopy (TEM): CPMV and CPMV-AUNP (10 μL of 0.1 mg/mL) were deposited onto Formvar carbon-coated copper grids (Electron Microscopy Sciences) for 2 min at room temperature. The grids were then washed twice with deionized water for 45 s and stained twice with 2% (w/v) uranyl acetate in deionized water for another 30 s. A Tecnai Spirit G2 transmission electron microscope was used to analyze the samples at 80 kV.

Size exclusion chromatography (SEC): CPMV and CPMV-AUNP (100 μL of 1 mg/mL) were loaded onto a Superose-6 increase column on the ÄKTA Explorer system (GE Healthcare). The column was analyzed using a flow rate of 0.5 mL/min in KP buffer pH 7.0.

CPMV and CPMV-AUNP therapy in a tumor mouse model: All animal studies were conducted upon approval and in accordance with the University of California, San Diego Institutional Animal Care and Use Committee (IACUC) guidelines. Six-week-old female C57BL/6J mice were purchased from The Jackson Laboratory. ID8-vegfA-defb29 murine ovarian serous carcinoma cell line was cultured at 37° C. in RPMI 1640 complete media (Sigma Aldrich) supplemented with 10% (v/v) fetal bovine serum (Atlanta Biologicals), 1 mmol/L sodium pyruvate (Sigma Aldrich), 1% (v/v) penicillin/streptomycin mixture (Sigma Aldrich), and 2 mmol/L L-glutamine (Sigma Aldrich). Cells were harvested and washed with RPMI medium. Eight-week-old mice were challenged with 2×10⁶ tumor cells in 400 μL sterile 1×PBS intraperitoneally (IP) on day 0. Mice were then randomly assigned into treatment groups and treatment was given by IP administration on day 8, 15, 22, 29, 36, and 42. The following treatment arms were assigned (200 μL IP injection): PBS control (n=5), 1 μg AUNP (n=3), 100 μg CPMV (n=5), 100 μg CPMV-AUNP (n=7), 100 μg CPMV+1 μg AUNP physical mixture (n=7). Mice were weighed regularly to monitor ascites formation and measure tumor burden. Mice were euthanized with carbon dioxide when they reached the humane endpoint of 35 g of weight, indicating significant ascites formation. Tumor burden was measured by increase in body weight and data were analyzed and plotted using Graphpad Prism software.

### Results

### 2.1. Preparation and Characterization of the CPMV-AUNP Formulation

CPMV-AUNP was obtained by conjugating SNTSESF (SEQ ID NO: 1) via an intervening linker GSGGGSGG (SEQ ID NO: 5) and carboxy-terminal cysteine residue to CPMV using an AUR-7 peptide [17]

(SEQ ID NO: 2)
SNTSESFGSGGGSGGC.

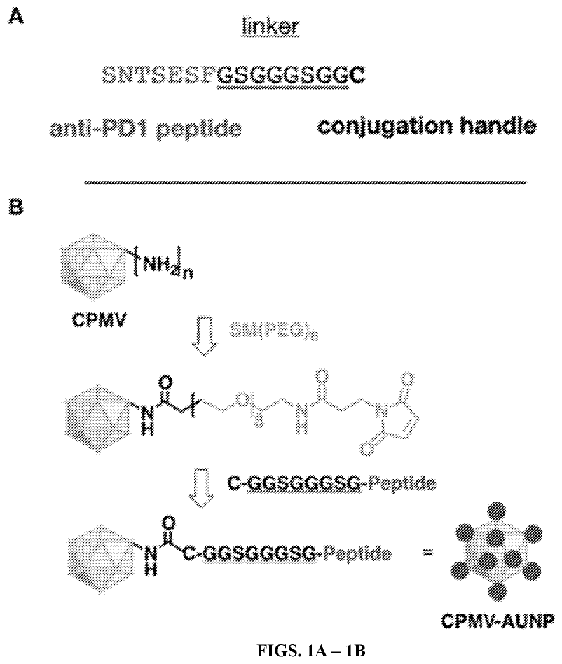

to CPMV using a bi-functional N-hydroxysuccinimide (NHS)-PEG8-maleimide (SM-PEG₈) linker (FIG. 1). CPMV nanoparticles present 300 addressable, solvent-exposed lysine side chains for functionalization [18]. The NHS arm of the SM-PEG₈ linker connects to CPMV to form a stable amide bond and the maleimide-functional group then allows conjugation of the cysteine-terminated anti-PD-1 peptide

(SEQ ID NO: 2)
SNTSESFGSGGGSGGC.

The resulting CPMV-AUNP is then purified by spin filtration using 100 kDa-cut off centrifugal devices and characterized using a combination of native and denaturing gel electrophoresis, transmission electron microscopy (TEM) size exclusion chromatography (SEC) to confirm structural integrity and to confirm assay the degree of peptide display.

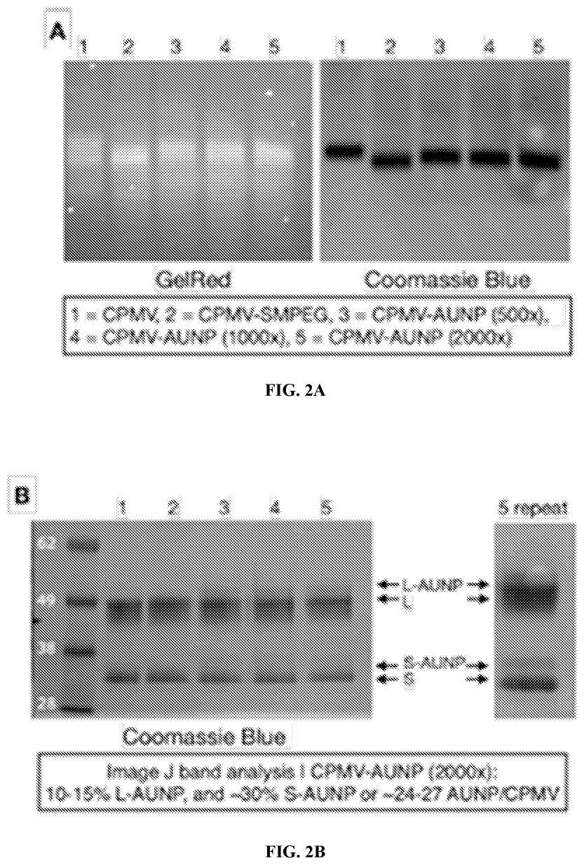

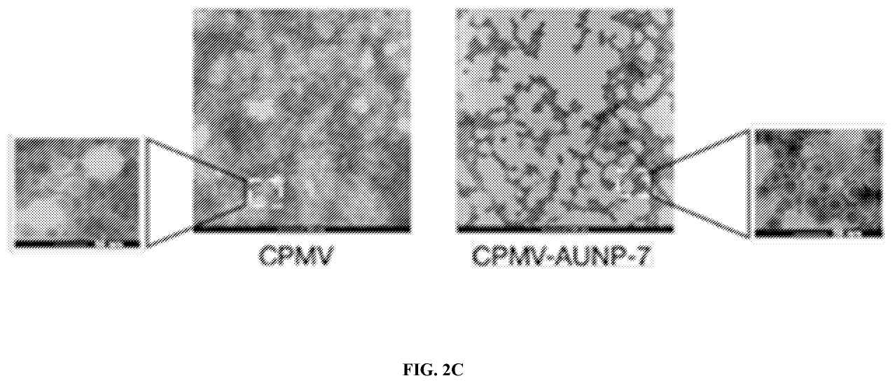

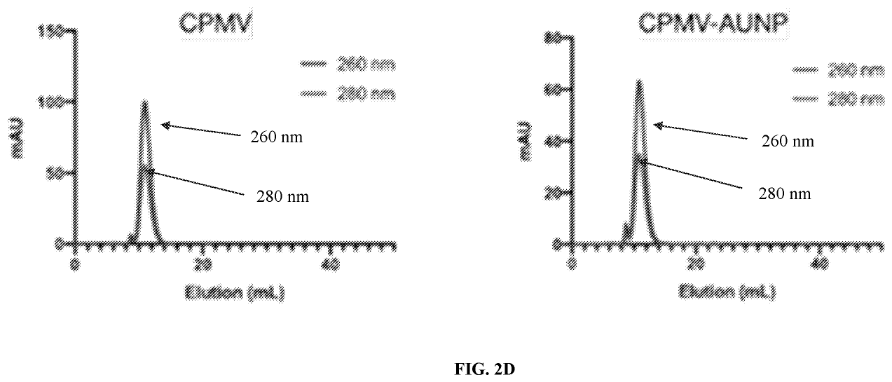

First, CPMV vs. CPMV-SMPEG vs. CPMV-AUNP formulations were analyzed by native agarose gel electrophoresis (FIG. 2A) and after electrophoretic separation gels were stained with GelRed and imaged under UV light to detect the encapsidated nucleic acids and then stained with Coomassie Brilliant Blue and imaged under white light to detect the protein capsids. Native agarose gel electrophoresis indicates that all samples analyzed remained intact: the RNA and protein bands co-localize and there is no evidence of broken particles, free RNA or aggregation. The CPMV-SMPEG formulation has higher electrophoretic mobility toward the anode, which is in agreement with positive charge reduction as a neutral linker (SM-PEG₈) is conjugated to surface lysines. Mobility of CPMV-AUNP conjugates is reduced compared to the CPMV-SMPEG formulation and this can be explained by an interplay of charge and increased molecular weight.

(SEQ ID NO: 2)
SNTSESFGSGGGSGGC.

has a molecular weight of 1.4 kDa and pI of 3.3 and a net negative charge of −1 (peptide calculator, https://www.bachem.com/). Applicant tested conjugation of

(SEQ ID NO: 2)
SNTSESFGSGGGSGGC.

to CPMV using an excess of 500, 1000, and 2000 M peptide to CPMV (FIG. 2A, lanes 3-5). Data indicate increasing mobility toward the anode with increased molecular excess used, which may indicate that the higher molar excess used led to increased peptide conjugation therefore increasing the negative charge of CPMV-AUNP and its mobility toward the anode. Second, SDS-PAGE confirmed the covalent attachment of the anti-PD-1 peptide to CPMV. In addition to the small (S, 24 kDa) and large (L, 42 kDa) CPMV coat proteins, higher molecular weight bands were detected for the CPMV-AUNP formulations (FIG. 2B). Band analysis using ImageJ software (https://imagej.nih.gov/ij/) indicated that 24-27 AUNP were conjugated to CPMV when using a 2000 M excess of peptide. Data were reproducible under these bioconjugate reaction conditions and chose the 2000 M excess for any experiments going forward.

To further verify the structural integrity of CPMV-AUNP, TEM imaging of negatively-stained samples was conducted and imaging confirmed the presence of intact, monodisperse nanoparticles measuring 30 nm in size (FIG. 2C). There was no apparent difference comparing CPMV vs. CPMV-AUNP. This was further validated by SEC which indicated intact CPMV and CPMV-AUNP eluting from the Superose 6 increase column; RNA (detected at 260 nm) and protein (detected as 280 nm) co-elute at ˜ 12 mL with A260:280 nm ratio of ˜ 1.8 which indicates presence of intact CPMV particles (FIG. 2D). Broken or disassembled coat protein units or particle aggregation was not apparent from SEC measurements, and this is consistent with agarose gels and TEM data.

### 2.2. In Vivo Efficacy of the CPMV-AUNP Formulation Against Ovarian Cancer

To assay efficacy of the CPMV-AUNP nanoparticle, Applicant used a mouse model of serous ovarian cancer. Specifically, Applicant used hyper-aggressive ID8defb29/vegf cells administered intraperitoneally (IP) in C57BL/6J mice because the histopathology and immunological response of such tumors closely resemble human disease [19]. Only female animals were used because Applicant is targeting ovarian cancer. Applicant used luciferase-labeled ID8defb29/vegf cells, allowing us to quantify the disease burden and establishment of disease (data not shown). Imaging is only informative at early time points, before the development of ascites, therefore Applicant used imaging to confirm successful tumor cell injection and tumor formation but then monitored body weight to assess tumor burden and 35 grams body weight was defined as the endpoint for the study. After tumor inoculation, mice were randomly assigned into treatment groups and treatment was given by IP administration on day 8, 15, 22, 29, 36, and 42. The following treatment arms were assigned (200 μL IP injection): PBS control (n=5), 1 μg AUNP (n=3), 100 μg CPMV (n=5), 100 μg CPMV-AUNP (n=7), 100 μg CPMV+1 μg AUNP physical mixture (n=7).

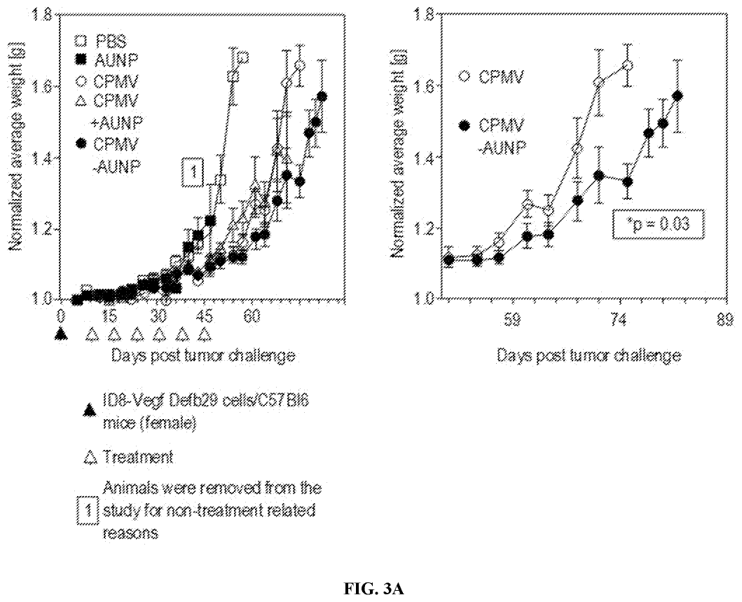

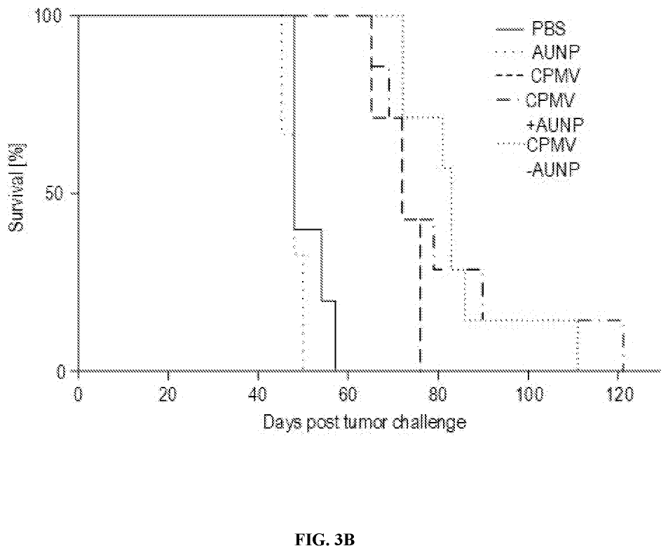

All control animals (PBS-treated) reached endpoint at day 57 (FIGS. 3A-3B). As previously demonstrated [10], weekly treatment using CPMV at a dose of 100 μg shows efficacy against these aggressive and disseminated ovarian tumors resulting in prolonged survival: 4 out of 6 animals reached endpoint at day 71 and 2 animals reached endpoint at day 75 (FIGS. 3A-3B). Free AUNP peptide has no apparent effect at the weekly dosing using 1 μg (this dose was matched to the dose of peptide administered when conjugated to CPMV-AUNP). Animals in this group had to be removed from the study due to non-treatment related reasons at day 47. However up to this day the tumor burden as measured by increase in body weight matched closely the PBS control group indicating at this dose the minimal sequence of SNTSESF (SEQ ID NO: 1) is not effective as solo-treatment arm (FIGS. 3A-3B). Similar data indicate that the physical mixture of CPMV and the AUNP peptide did not improve efficacy beyond that observed for CPMV alone. In contrast increased efficacy was apparent for the CPMV-AUNP group: only 1 animal reached endpoint at day 75 (all CPMV animals reached endpoint at this timepoint); 2 animals in the CPMV-AUNP remained in the study until day 85 with the last animal reaching endpoint at day 99 (FIGS. 3A-3B). Divergence of the tumor growth curves (measured based on body weight increase due to ascites formation) is apparent from day 45 with statistical significance observed from day 75 (FIG. 3A and Table 1). While the increase in tumor efficacy is moderate, it is interesting that the minimal peptide SNTSESF (SEQ ID NO: 1) conjugated at rather sparse density (<30 copies per 30 nm-sized nanoparticle) potentiates the potency of CPMV alone. This was only observed for the conjugated formulation and not when SNTSESF (SEQ ID NO: 1) was added as free peptide. This could be explained by the differences in the in vivo fates of the nanoparticle formulation vs. the free peptide, the latter likely experiences rapid wash out effects from the tumor, while the larger nanoparticles are expected to exhibit prolonged tumor residence and altered intratumoral distribution.

TABLE 1
Table 1: Statistics of data presented in FIG. 3A; statistical
significance was calculated by one-way ANOVA and TTEST.

| | | P Value |
|---|---|---|
| | Summary | One-way Anova |
| PBS vs. CPMV | *** | 0.0001 |
| PBS vs. CPMV − AUNP | *** | 0.0007 |
| PBS vs. CPMV + AUNP | ** | 0.0021 |
| PBS vs. AUNP | ns | 0.9985 |
| CPMV vs. CPMV − AUNP | ns | 0.9992 |
| CPMV vs. CPMV + AUNP | ns | 0.9624 |

Day 75: CPMV vs. CPMV − AUNP * P value 0.03 (TTEST)

### DISCUSSION

Applicant reports herein the design of CPMV-AUNP nanoparticles and demonstrate efficacy against tumors in a mouse model of disseminated and aggressive ovarian cancer. Ovarian cancer is the foremost cause of gynecological cancer and a major cause of cancer death in women [20]. A clinical challenge is that the disease is often not diagnosed prior to stage III (metastasis to peritoneal cavity) or stage IV (metastasis outside of peritoneal cavity), so most patients present with a highly metastatic disease that cannot be cured surgically. Surgical debulking followed by chemotherapy is the standard of care. Relapse occurs in 70-90% of stage III and 90-95% of stage IV [21]. Immunotherapy has now become established as the fourth pillar of cancer treatment and for some cancers has already significantly reduced mortality [22-24]. Immunotherapies such as in situ vaccination approaches as Applicant described herein hold tremendous potential to improve patient outcomes and safe lives of women with ovarian tumors.

Applicant previously reported efficacy of the CPMV in situ vaccine when used as solo-therapy as well as in combination with chemotherapy [25], radiation and immune checkpoint therapy [15]. With regards to CPMV and immune checkpoint therapy combinations, this is a particularly powerful combination: In situ vaccination with CPMV increases antigen specific effector T cells and this is how it generates systemic resistance to the treated tumor. To be effective, such vaccine fueled immune activation also requires release of the immunosuppressive brakes that are upregulated in aggressive tumors and in response to the immunotherapy. Immune checkpoint blocking antibodies are effective at removing inhibitory signals but as monotherapy are efficient only against highly immunogenic tumors; expanded efficacy in non-immunogenic tumors can be achieved through immunogenic interventions such as in situ vaccines to enable a tumor antigen-specific cytotoxic T lymphocyte response. Indeed Applicant have shown that combination of CPMV with anti-PD-1 antibodies results in improved anti-tumor efficacy in tumor mouse models [15].

Here Applicant extended this work and combined CPMV with a small molecule anti-PD-1 peptide; specifically Applicant chose the minimal sequence SNTSESF (SEQ ID NO: 1) of the previously described AUNP-12 peptide (also known as NP-12) shown to inhibits PD-1 function [17]. While the full-length branched peptide results in optimal efficacy, the minimal sequence SNTSESF (SEQ ID NO: 1) was shown to have surprisingly high activities [27]. Indeed, conjugation and multivalent display of SNTSESF (SEQ ID NO: 1) on CPMV-AUNP resulted in moderately enhanced efficacy vs. CPMV alone. No improvement in efficacy was observed when CPMV was mixed with free peptide. The fact that free peptide showed no efficacy and conferred no improvement when mixed with CPMV may be explained by the dose and administration schedule. Here Applicant based dose and administration schedule on the typical CPMV schedule; weekly treatment using 100 μg CPMV; for every 100 μg CPMV-AUNP 1 μg SNTSESF (SEQ ID NO: 1) peptide were delivered. Formulation chemistry can also be modified: ˜ 25 peptide per CPMV were presented using a two-step bioconjugation protocol that utilizes a cysteine-terminated peptide and SM-PEG linker. CPMV offers 300 addressable surface lysine side chains and therefore there is room for improvement. Increased molar excess of peptide and linker may yield increased bioconjugation efficiency—as an alternative one may consider orthogonal reactions such as Cu(I)-catalyzed azide-alkyne cycloadditions [28,29] or hydrazone-based coupling strategies [30]. Nevertheless, the fact that even a suboptimal formulation yields increased efficacy is promising. The increased tumor residence time of the much larger nanoparticle vs. the low molecular weight free peptide likely explains the observed efficacy. While the free peptide likely experiences rapid tumor wash out effects and proteolytic degradation, the nanoparticle conjugate offers stability. Similar phenomena were reported for other immunotherapy strategies; for example, CpGs which act as TLR-9 agonists show increased potency when delivered by a nanoparticle and this has been attributed to prolonged tumor residence and altered intratumoral distribution [31].

Mammalian in situ vaccination approaches and oncolytic viral therapies, e.g. Imlygic (Amgen) [6] have already made headways; however plant viral nanoparticles offer advantages compared to mammalian vectors or synthetic nanoparticle technologies: (1) Production through farming in plants is highly scalable for commercialization and the plant viruses can be stably stored (and are stable without cold chain requirements). (2) Plant viruses do not infect or replicate in mammalian cells, thus adding another layer of safety compared to oncolytic viral therapies. (3) The materials are uniform and monodisperse, a level of quality control/assurance difficult to achieve using synthetic approaches. (4) Lastly, it is important to understand that the CPMV cancer immunotherapy is conceptually distinct from oncolytic cancer therapy: oncolytic viruses (including TVEC) function by targeting and killing cancer cells—however, CPMV targets innate immune cells to prime systemic anti-immunity (adaptive arm). A particular advantage is—because CPMV targets the innate immune system—presence of carrier-specific antibodies (which may be formed during repeat treatment schedules) are not neutralizing; rather presence of anti-CPMV antibodies enhances the potency of the CPMV in situ vaccine over time. This makes sense; CPMV does not target cancer cells, but immune cells. Therefore, the presence of antibodies against CPMV opsonizes the nanoparticle enhancing its uptake in innate immune cells, thus boosting the anti-tumor response [32].

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims. All references are herein incorporated in their entirety for any and all purposes.

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

Partial Sequence Listing

| | |
|---|---|
| SEQ ID NO. 1: | SNTSESF |
| SEQ ID NO. 2: | SNTSESFGSGGGSGGC |
| SEQ ID NO. 3: | NYSKPTDRQYHF |
| SEQ ID NO. 4: | SNTSESFKFRVTQLAPKAQIKE |
| SEQ ID NO. 5: | GSGGGSGG |
| SEQ ID NO. 6: | GSGGGSGGC |

### REFERENCES

[1] D. S. Chen, I. Mellman, Oncology meets immunology: the cancer-immunity cycle, Immunity 39(1) (2013) 1-10.
[2] W. Alexander, The Checkpoint Immunotherapy Revolution: What Started as a Trickle Has Become a Flood, Despite Some Daunting Adverse Effects; New Drugs, Indications, and Combinations Continue to Emerge, P T 41 (3) (2016) 185-91.
[3] M. D. Hellmann, C. F. Friedman, J. D. Wolchok, Combinatorial Cancer Immunotherapies, Adv Immunol 130 (2016) 251-77.
[4] L. A. Emens, P. A. Ascierto, P. K. Darcy, S. Demaria, A. M. M. Eggermont, W. L. Redmond, B. Seliger, F. M. Marincola, Cancer immunotherapy: Opportunities and challenges in the rapidly evolving clinical landscape, Eur J Cancer 81 (2017) 116-129.
[5] M. R. Sheen, S. Fiering, In situ vaccination: Harvesting low hanging fruit on the cancer immunotherapy tree, Wiley Interdiscip Rev Nanomed Nanobiotechnol 11 (1) (2019) e1524.
[6] P. F. Ferrucci, L. Pala, F. Conforti, E. Cocorocchio, Talimogene Laherparepvec (TVEC): An Intralesional Cancer Immunotherapy for Advanced Melanoma, Cancers (Basel) 13 (6) (2021).
[7] C. Mao, V. Beiss, J. Fields, N. F. Steinmetz, S. Fiering, Cowpea mosaic virus stimulates antitumor immunity through recognition by multiple MYD88-dependent toll-like receptors, Biomaterials 275 (2021) 120914.
[8] A. A. Murray, C. Wang, S. Fiering, N. F. Steinmetz, In Situ Vaccination with Cowpea vs Tobacco Mosaic Virus against Melanoma, Mol Pharm 15 (9) (2018) 3700-3716.
[9] P. H. Lizotte, A. M. Wen, M. R. Sheen, J. Fields, P. Rojanasopondist, N. F. Steinmetz, S. Fiering, In situ vaccination with cowpea mosaic virus nanoparticles suppresses metastatic cancer, Nat Nanotechnol 11 (3) (2016) 295-303.
[10] C. Wang, S. Fiering, N. F. Steinmetz, Cowpea Mosaic Virus Promotes Anti-Tumor Activity and Immune Memory in a Mouse Ovarian Tumor Model, Advanced Therapeutics (2019) 1900003.
[11] A. Kerstetter-Fogle, S. Shukla, C. Wang, V. Beiss, P. L. R. Harris, A. E. Sloan, N. F. Steinmetz, Plant Virus-Like Particle In Situ Vaccine for Intracranial Glioma Immunotherapy, Cancers (Basel) 11 (4) (2019).
[12] P. J. Hoopes, C. M. Mazur, B. Osterberg, A. Song, D. J. Gladstone, N. F. Steinmetz, F. A. Veliz, A. A. Bursey, R. J. Wagner, S. N. Fiering, Effect of intratumoral magnetic nanoparticle hyperthermia and viral nanoparticle immunogenicity on primary and metastatic cancer, Proc SPIE Int Soc Opt Eng 10066 (2017).
[13] P. J. Hoopes, K. L. Moodie, A. A. Petryk, J. D. Petryk, S. Sechrist, D. J. Gladstone, N. F. Steinmetz, F. A. Veliz, A. A. Bursey, R. J. Wagner, A. Rajan, D. Dugat, M. Crary-Burney, S. N. Fiering, Hypo-fractionated Radiation, Magnetic Nanoparticle Hyperthermia and a Viral Immunotherapy Treatment of Spontaneous Canine Cancer, Proc SPIE Int Soc Opt Eng 10066 (2017).
[14] P. J. Hoopes, R. J. Wagner, K. Duval, K. Kang, D. J. Gladstone, K. L. Moodie, M. Crary-Burney, H. Ariaspulido, F. A. Veliz, N. F. Steinmetz, S. N. Fiering, Treatment of Canine Oral Melanoma with Nanotechnology-Based Immunotherapy and Radiation, Mol Pharm (2018).
[15] C. Wang, N. F. Steinmetz, A Combination of Cowpea mosaic virus and Immune Checkpoint Therapy Synergistically Improves Therapeutic Efficacy in Three Tumor Models, Advanced Functional Materials (2020) https://doi.org/10.1002/adfm.202002299.
[16] H. N. Chang, B. Y. Liu, Y. K. Qi, Y. Zhou, Y. P. Chen, K. M. Pan, W. W. Li, X. M. Zhou, W. W. Ma, C. Y. Fu, Y. M. Qi, L. Liu, Y. F. Gao, Blocking of the PD-1/PD-L1 Interaction by a D-Peptide Antagonist for Cancer Immunotherapy, Angew Chem Int Ed Engl 54 (40) (2015) 11760-4.
[17] P. G. Sasikumar, R. K. Ramachandra, S. Adurthi, A. A. Dhudashiya, S. Vadlamani, K. Vemula, S. Vunnum, L. K. Satyam, D. S. Samiulla, K. Subbarao, R. Nair, R. Shrimali, N. Gowda, M. Ramachandra, A Rationally Designed Peptide Antagonist of the PD-1 Signaling Pathway as an Immunomodulatory Agent for Cancer Therapy, Mol Cancer Ther 18 (6) (2019) 1081-1091.
[18] A. Chatterji, W. Ochoa, M. Paine, B. R. Ratna, J. E. Johnson, T. Lin, New addresses on an addressable virus nanoblock: uniquely reactive Lys residues on cowpea mosaic virus, Chem Biol 11 (6) (2004) 855-63.
[19] J. R. Conejo-Garcia, F. Benencia, M. C. Courreges, E. Kang, A. Mohamed-Hadley, R. J. Buckanovich, D. O. Holtz, A. Jenkins, H. Na, L. Zhang, D. S. Wagner, D. Katsaros, R. Caroll, G. Coukos, Tumor-infiltrating dendritic cell precursors recruited by a beta-defensin contribute to vasculogenesis under the influence of Vegf-A, Nat Med 10 (9) (2004) 950-8.
[20] M. A. Lisio, L. Fu, A. Goyeneche, Z. H. Gao, C. Telleria, High-Grade Serous Ovarian Cancer: Basic Sciences, Clinical and Therapeutic Standpoints, Int J Mol Sci 20 (4) (2019).
[21] D. D. Bowtell, S. Bohm, A. A. Ahmed, P. J. Aspuria, R. C. Bast, Jr., V. Beral, J. S. Berek, M. J. Birrer, S. Blagden, M. A. Bookman, J. D. Brenton, K. B. Chiappinelli, F. C. Martins, G. Coukos, R. Drapkin, R. Edmondson, C. Fotopoulou, H. Gabra, J. Galon, C. Gourley, V. Heong, D. G. Huntsman, M. Iwanicki, B. Y. Karlan, A. Kaye, E. Lengyel, D. A. Levine, K. H. Lu, I. A. McNeish, U. Menon, S. A. Narod, B. H. Nelson, K. P. Nephew, P. Pharoah, D. J. Powell, Jr., P. Ramos, I. L. Romero, C. L. Scott, A. K. Sood, E. A. Stronach, F. R. Balkwill, Rethinking ovarian cancer II: reducing mortality from high-grade serous ovarian cancer, Nat Rev Cancer 15 (11) (2015) 668-79.
[22] H. Zhang, J. Chen, Current status and future directions of cancer immunotherapy, J Cancer 9 (10) (2018) 1773-1781.
[23] M. Chiriva-Internati, A. Bot, A new era in cancer immunotherapy: discovering novel targets and reprogramming the immune system, Int Rev Immunol 34 (2) (2015) 101-3.
[24] A. Dart, New targets for cancer immunotherapy, Nat Rev Cancer 18 (11) (2018) 667.
[25] H. Cai, C. Wang, S. Shukla, N. F. Steinmetz, Cowpea Mosaic Virus Immunotherapy Combined with Cyclophosphamide Reduces Breast Cancer Tumor Burden and Inhibits Lung Metastasis, Adv Sci (Weinh) 6 (16) (2019) 1802281.
[26] P. J. Hoopes, R. J. Wagner, K. Duval, K. Kang, D. J. Gladstone, K. L. Moodie, M. Crary-Burney, H. Ariaspulido, F. A. Veliz, N. F. Steinmetz, S. N. Fiering, Treatment of Canine Oral Melanoma with Nanotechnology-Based Immunotherapy and Radiation, Mol Pharm 15 (9) (2018) 3717-3722.
[27] E. Differding, Aunp-12-a Novel Peptide Therapeutic Targeting Pd-1 Immune Checkpoint Pathway for Cancer Immunotherapy@Structure Activity Relationships & Peptide/Peptidomimetic Analogs, 2014.
[28] S. I. Presolski, V. Hong, S. H. Cho, M. G. Finn, Tailored ligand acceleration of the Cu-catalyzed azide-alkyne cycloaddition reaction: practical and mechanistic implications, J Am Chem Soc 132 (41) (2010) 14570-6.
[29] V. Hong, S. I. Presolski, C. Ma, M. G. Finn, Analysis and optimization of copper-catalyzed azide-alkyne cycloaddition for bioconjugation, Angew Chem Int Ed Engl 48 (52) (2009) 9879-83.
[30] F. M. Brunel, J. D. Lewis, G. Destito, N. F. Steinmetz, M. Manchester, H. Stuhlmann, P. E. Dawson, Hydrazone ligation strategy to assemble multifunctional viral nanoparticles for cell imaging and tumor targeting, Nano Lett 10 (3) (2010) 1093-7.
[31] M. O. Mohsen, D. E. Speiser, A. Knuth, M. F. Bachmann, Virus-like particles for vaccination against cancer, Wiley Interdiscip Rev Nanomed Nanobiotechnol (2019) e1579.
[32] S. Shukla, C. Wang, V. Beiss, N. F. Steinmetz, Antibody Response against Cowpea Mosaic Viral Nanoparticles Improves In Situ Vaccine Efficacy in Ovarian Cancer, ACS Nano 14 (3) (2020) 2994-3003.
[33] C. E. Boone, L. Wang, A. Gautam, I. G. Newton, N. F. Steinmetz, Combining nanomedicine and immune checkpoint therapy for cancer immunotherapy, Wiley Interdiscip Rev Nanomed Nanobiotechnol (2021) e1739.
[34] J. Wellink, Comovirus isolation and RNA extraction, Methods Mol Biol 81 (1998) 205-9.

1. A nanoparticle comprising a cowpea mosaic virus (CPMV) or cowpea chlorotic mottle virus (CCMV) and a peptide that binds to an immune checkpoint or its ligand, optionally PD-1 or PD-L1, optionally wherein the peptide further comprises a linker peptide, optionally wherein the CPMV or CCMV further comprise an exposed lysine side chain, and optionally wherein the nanoparticle has an average diameter of from about 10 to about 50 nm.

2. The nanoparticle of claim 1, wherein the peptide comprises the amino acid of any one or more of SNTSESF (SEQ ID NO: 1) or SNTSESFGSGGGSGGC (SEQ ID NO: 2), NYSKPTDRQYHF (SEQ ID NO: 3), SNTSESFKFRVTQLAPKAQIKE (SEQ ID NO: 4) or an equivalent thereof having at least 70% identity or similarity to SEQ ID NO: 1, SEQ ID NO: 2, SEQ ID NO: 3, or an equivalent thereof having at least 70% identity or similarity to SEQ ID NO: 4 from which the peptide SNTSESF (SEQ ID NO: 1) branches at the underlined lysine residue.

3-5. (canceled)

6. The nanoparticle of claim 1, wherein the linker peptide comprises GSGGGSGGX (SEQ ID NO: 5) or an equivalent thereof, wherein X is an optional carboxy-terminal cysteine residue (C) (SEQ ID NO: 6).

7. (canceled)

8. The nanoparticle of claim 1, wherein the exposed lysine side chain is conjugated to a maleimide-PEG-NHS linker by a reaction at the N-hydroxysuccinimide (NHS) ester of the PEG linker.

9. The nanoparticle of claim 1, wherein the peptide is chemically conjugated to the CMPV or CCMV, optionally wherein the peptide is chemically conjugated through a maleimide-PEG-NHS reaction between the c-terminal cysteine of the peptide and the maleimide of the PEG linker.

10. (canceled)

11. The nanoparticle of claim 1, wherein the peptide is an azide/alkyne modified peptide and further wherein said peptide is conjugated to the nanoparticle through a click chemistry reaction with an azide/alkyne modified CPMV or CCMV.

12. The nanoparticle of claim 1, wherein the peptide is added to the CMPV or CCMV as an N-terminal genetic fusion in a CPMV or CCMV plasmid.

13-14. (canceled)

15. A polynucleotide encoding the nanoparticle of claim 1 or its complement, wherein the polynucleotide is RNA or DNA.

16. (canceled)

17. A vector comprising the polynucleotide of claim 15, optionally comprising a promoter and/or an enhancer, optionally wherein the vector is a plasmid.

18. (canceled)

19. A host cell comprising the nanoparticle of claim 1, wherein the host cell is a prokaryotic cell, a eukaryotic cell, a plant cell, or a bacterial cell.

20-23. (canceled)

24. A composition comprising the nanoparticle of claim 1, optionally a carrier, and further optionally additional therapeutic agents, and optionally wherein the composition is lyophilized.

25-27. (canceled)

28. A method for inducing an immune response in a subject in need thereof comprising administering to the subject the nanoparticle of claim 1.

29. A method for inhibiting the growth of a cancer cell comprising contacting the cell with the nanoparticle of claim 1, wherein the contacting is in vitro or in vivo, and optionally wherein the cancer cell expressed PD-1.

30. (canceled)

31. A method for treating cancer in a subject in need thereof, comprising administering to the subject: the nanoparticle of claim 1 and optionally a different cancer therapy, wherein the cancer is a primary or a metastatic cancer, wherein the subject is a mammal or a human, and optionally wherein the cancer expresses PD-1.

32-38. (canceled)

39. The method of claim 31, wherein the administering comprises one or more of the following: intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmucosal, or inhalation delivery.

40. (canceled)

41. The method of claim 31 further comprising administering to the subject an anti-tumor or other therapy to the benefit of the subject other than the nanoparticle disclosed herein, optionally wherein the anti-tumor therapy comprises tumor resection, further optionally wherein the treatment further comprises the nanoparticle of claim 1 delivered or administered into a cavity formed by tumor resection (intracavity delivery) or directly into a tumor prior to tumor resection (intratumoral delivery).

42-43. (canceled)

44. The method of claim 31, wherein the nanoparticle, plurality, vector, host cell, or composition are administered from 10 times a day to once a month for a period of from one day to 20 months.

45. A method of altering an immune cell profile in a tumor of a subject comprising the nanoparticle of claim 1, wherein the subject is a mammal or a human, and optionally wherein the tumor expresses PD-1.

46. (canceled)

47. A kit comprising the nanoparticle of claim 1 and optional additional therapeutic compositions or methods.

48. (canceled)

49. A nanoparticle comprising a CPMV and an anti-PD-1 peptide comprising the sequence SNTSESF (SEQ ID NO: 1) or an equivalent thereof having at least 70% identity thereto.
